(19) United States
(12) Patent Application Publication
(10) Pub. No.: US 2020/0284790 A1
(43) Pub. Date: Sep. 10, 2020

(54) BACTERIA CAUSING SEXUALLY-TRANSMITTED DISEASES AND IMMUNE T-CELL DETECTION

(71) Applicant: Essenlix Corporation, Monmouth Junction, NJ (US)

(72) Inventors: Stephen CHOU, Princeton, NJ (US); Wei DING, East Windsor, NJ (US); Ji LI, Princeton, NJ (US); Sheng-Jian CAI, Bridgewater, NJ (US)

(73) Assignee: Essenlix Corporation, Monmouth Junction, NJ (US)

(21) Appl. No.: 16/759,050

(22) PCT Filed: Oct. 26, 2018
(86) PCT No.: PCT/US18/57872

§ 371 Date: Apr. 24, 2020

Related U.S. Application Data
(60) Provisional application No. 62/577,440, filed on Oct. 26, 2017.

(51) Int. Cl. G01N 33/543; G01N 33/577; G01N 33/50; G01N 33/58; G01N 21/64

(52) U.S. Cl. CPC G01N 33/54386; G01N 33/577; G01N 33/5094; G01N 33/582; G01N 21/6428; G01N 2458/00; G01N 21/554; G01N 2333/70517; G01N 2333/70514; G01N 2333/7051; G01N 2333/22; G01N 2333/20; G01N 2021/6439; G01N 2333/295

(57) ABSTRACT

Methods and devices are provided for rapid detection in a sample of bacteria that cause sexually transmitted diseases (chlamydia, gonorrhea, or syphilis). Methods and devices are also provided for the rapid detection of immune cells (CD3, CD4 and CD8 cells).

### CROSS REFERENCING

This application is a National Stage entry (§ 371) application of International Application No. PCT/US18/57872, filed on Oct. 26, 2018, which claims the benefit of U.S. Provisional Patent Application No. 62/577,440, filed on Oct. 26, 2017, the contents of which are relied upon and incorporated herein by reference in their entirety.

The entire disclosure of any publication or patent document mentioned herein is entirely incorporated by reference.

### FIELD

Among other things, the present invention is related to devices and methods of performing biological and chemical assays, such as immunoassays.

### BACKGROUND

Bacterial infections with Chlamydia trachomatis (causes Chlamydia)), Neisseria gonorrhoeae (causes Gonorrhea) or Treponema pallidium (causes Syphilis) are common sexually transmitted diseases (STDs) that can cause serious, permanent damage to a person's health. The Centers for Disease Control and Prevention (CDC) estimates that more than two and a half million Americans are infected with chlamydia every year. STDs are especially common in sexually active people aged 15 to 24. Many individuals with STDs don't have symptoms, so the CDC and other health organizations recommend regular screening for groups at higher risk. Traditional testing for many of these bacteria such as chlamydia (e.g., cell culture), however, is time consuming (5-7 days) and difficult to conduct.

Immune T-cells that plays critical roles in cell-mediated immunity. Several subsets of T cells each have a distinct functions. CD4 (cluster of differentiation 4) is a glycoprotein found on the surface of immune cells such as T helper cells, monocytes, macrophages, and dendritic cells. CD4+ cells secrete small proteins called cytokines that regulate or assist in the active immune response. The number of CD4 expressing cells have been widely accepted as an indicator of HIV (human immunodeficiency virus) infection and AIDS (acquired immune deficiency syndromes). CD8+ T cells express the CD8 glycoprotein at their surfaces which recognize antigen associated with MHC class I molecules. CD8 cells destroy virus-infected cells and tumor cells, and are also implicated in transplant rejection.

There is a continuing need for a faster and simpler test for chlamydia, and a rapid and reliable test for CD3+, CD8+ and CD4+ cells. The present invention provides devices and methods to perform assays that can be used to detect bacteria that causes STDs and/or quantify T-cells (such as CD4 expressing cells) in a sample, with high speed and efficiency.

### SUMMARY

In one aspect, the present invention provides a method, comprising the steps of providing a device comprising a first plate and a second plate, said first plate and/or said second plate comprises, on its inner surface, a sample contact area that is configured to contact a sample, wherein the sample contains or suspected of containing bacteria that cause sexually transmitted diseases (STDs); depositing the sample onto the sample contact area; adding a staining medium to the deposited sample to form a mixture, wherein the staining medium comprises an antibody specific to the bacteria; compressing the first plate with the second plate so that at least part of the mixture forms a thin layer; incubating the mixture so that the antibody binds to the STD-causing bacteria and yields a signal; and detecting the signal, wherein the detected signal is indicative of the presence of bacteria in the sample.

The method of any embodiment of the present disclosure, wherein the bacteria that cause STD is selected from the group consisting of Chlamydia trachomatis, Neisseria gonorrhoeae, and Treponema pallidium.

The method of any embodiment of the present disclosure, wherein the incubating step performed for about 60 seconds or less.

The method of any embodiment of the present disclosure, wherein the incubating step performed for about 30 seconds or less.

The method of any embodiment of the present disclosure, wherein the incubating step is performed for about 15 seconds or less.

The method of any embodiment of the present disclosure, wherein the antibody is fluorescently labeled.

The method of any embodiment of the present disclosure, wherein the thin layer has a uniform thickness that is less than 100 μm.

The method of any embodiment of the present disclosure, wherein the thin layer has a uniform thickness that is less than 50 μm.

The method of any embodiment of the present disclosure, wherein the thin layer has a uniform thickness that is about 30 μm or less.

The method of any embodiment of the present disclosure, wherein the signal is detected by imaging the incubated sample step (f).

In one aspect, the present invention provides a method, comprising the steps of providing a device comprising a first plate and a second plate, one or both of the plates comprises, on its inner surface, a sample contact area that has a binding site, wherein the sample contact area is configured to contact a sample, wherein the sample contains or suspected of comprising bacteria that cause sexually transmitted diseases (STDs), and wherein the binding site comprises an immobilized capture antibody that binds to the bacteria in the sample; providing one or both of the plates comprising, on its inner surface, a sample contact area that has a storage site, wherein the storage site comprises a detection antibody that is capable of, upon contacting the sample, diffusing in the sample, and wherein the capture antibody and detection antibody bind to different sites on the bacteria to form a capture antibody-bacteria-detection antibody sandwich; depositing the sample onto one or both of the sample contact areas of the plates; bringing the two plates to a closed configuration, wherein, in the closed configuration, at least part of the deposited sample in (c) is confined between the sample contact areas of the two plates, and the first plate and the second plate has an average thickness in the range of 0.01 μm to 200 μm; and detecting a signal, wherein the signal is generated after the capture antibody-bacteria-detection antibody is formed, and the detected signal is indicative of the presence of bacteria that cause sexually transmitted diseases (STDs) in the sample.

The method of any embodiment of the present disclosure, wherein the sample is from a human.

The method of any embodiment of the present disclosure, wherein the bacteria is selected from the group consisting of Chlamydia trachomatis, Neisseria gonorrhoeae, and Treponema pallidium.

The method of any embodiment of the present disclosure, wherein the capture antibody has a capturing site that comprises a protein stabilizer.

The method of any embodiment of the present disclosure, wherein the storage site further comprises a protein stabilizer.

The method of any embodiment of the present disclosure, wherein the detection antibody comprises a fluorescent label.

The method of any embodiment of the present disclosure, wherein the sample between the two plates has a uniform thickness in the range of 0.5 μm to 50 μm.

The method of any embodiment of the present disclosure, wherein the sample between the two plates has a uniform thickness in the range of 1 μm to 35 μm.

The method of any embodiment of the present disclosure, further comprising the step (g): determining the presence or absence of STD-causing bacteria.

The method of any embodiment of the present disclosure, wherein the steps (a)-(e) are performed in less than 10 minutes.

The method of any embodiment of the present disclosure, wherein the steps (e)-(e) are performed in less than 3 minutes.

The method of any embodiment of the present disclosure, wherein the steps (a)-(e) are performed in less than 2 minutes.

The method of any embodiment of the present disclosure, wherein one or both of the sample contact areas comprise a plurality of spacers, wherein the plurality of spacers regulate the spacing between the sample contact areas of the plates when the plates are in the closed configuration.

The method of any embodiment of the present disclosure, wherein the first plate comprises a plurality of binding sites and the second plate comprises a plurality of corresponding storage sites, wherein each binding site faces a corresponding storage site when the plates are in the closed configuration.

The method and device of any embodiment of the present disclosure, wherein the detection antibody is dried on the storage site.

The method of any embodiment of the present disclosure, wherein the capture antibody at the binding site is on an amplification surface that amplifies an optical signal of the captured detection antibody.

The method of any embodiment of the present disclosure, wherein the capture antibody at the binding site are on an amplification surface that amplifies an optical signal of the captured detection antibody, wherein the amplification is proximity-dependent in that the amplification significantly reduced as the distance between the capture antibody and the detection antibody increases.

The method of any embodiment of the present disclosure, wherein the signal is detected by electrical means, optical means, or both.

The method of any embodiment of the present disclosure, wherein the signal is detected by fluorescence or SPR.

In one aspect, the present invention provides a method, comprising the steps of providing a providing a device comprising a first plate and a second plate, one or both of the plates comprises, on its inner surface, a sample contact area that has a binding site, wherein the sample contact area is configured to contact a liquid sample, wherein the liquid sample contains or is suspected of containing cells that express a biomarker, providing one or both of the plates comprising, on its inner surface, a sample contact area that has a storage site, wherein the storage site comprises a detection agent positioned therein, wherein the detection agent is configured to bind to the biomarker, depositing the sample onto one or both of the sample contact areas of the plates; wherein the deposited liquid sample is in contact with the detecting agent; bringing the two plates to a closed configuration, wherein, in the closed configuration, at least part of the deposited sample in (c) is confined between the sample contact areas of the two plates, and the first plate and the second plate has an average thickness in the range of 0.01 μm to 200 μm; incubating the deposited liquid sample for a period of time; quantifying the cells expressing the biomarker by imaging the deposited sample layer and counting the cells that expresses the biomarker.

In one aspect, the present invention provides a method, comprising providing a first plate and a second plate, wherein each plate comprises, on its respective inner surface, a sample contact area that is configured to contact a liquid sample, wherein a detecting agent is positioned on the sample contact area of one or both of the plates, and wherein the detecting agent is configured to specifically bind to the biomarker; depositing the sample onto the sample contact area, wherein the deposited sample comprises the cell that expresses the biomarker; pressing the first plate and the second plate to compress the deposited sample into a thin layer, which is at least partly confined by the two sample contact areas that face each other; incubating for a period of time that is about 60 seconds or less; and quantifying the cell expressing the biomarker by imaging the deposited sample layer and counting the cell expressing the biomarker.

In one aspect, the present invention provides a method, comprising providing a first plate and a second plate, each plate comprises, on its respective inner surface, a sample contact area that is configured to contact a blood sample, wherein a detecting agent is positioned on the sample contact area of one or both of the plates, and wherein the detecting agent is configured to specifically bind to an antigen selected from the group consisting of CD3, CD4 and CD8, depositing the blood sample in the sample contact area, wherein the blood sample comprises cells that express CD3, CD4, or CD8; pressing the first plate and the second plate to compress the blood sample into a thin layer, which is at least partly confined by the two sample contact areas that face each other; incubating for a period of time that is about 60 seconds or less; and quantifying the cells expressing CD3, CD4 or CD8 by imaging the compressed blood sample and counting the cells expressing CD3, CD4 or CD8.

In one aspect, the present invention provides an apparatus, comprising a first plate and a second plate, movable relative to each other into different configurations, including a closed configuration and an open configuration, wherein each plate comprises, on its respective inner surface, a sample contact area that is configured to contact a liquid sample that expresses or is expected to express a biomarker, wherein a detecting agent is positioned on one or both of the plates and is configured to specifically bind to the biomarker; and an adaptor that is configured to accommodate the first plate and second plate when in a closed configuration and be attachable to a mobile device, wherein the mobile device comprises an imager, the adaptor is configured to position the liquid sample in a field of view (FOV) of the imager when the adaptor is attached to the mobile device, and the imager is configured to capture images of the liquid sample, thereby detecting/measuring a signal that is generated by the binding of the biomarker with the detecting agent after the sample is incubated with the detecting agent for a period of time that is about 60 seconds or less.

The method or apparatus of any embodiment of the present disclosure, wherein the period of time that is about 30 seconds or less.

The method or apparatus of any embodiment of the present disclosure, with the proviso that the sample contact areas are not washed after the incubating step (d).

The method or apparatus of any embodiment of the present disclosure, wherein the detecting agent is an antibody.

The method or apparatus of any embodiment of the present disclosure, wherein the antibody is labeled with a fluorophore.

The method or apparatus of any embodiment of the present disclosure, wherein the detecting agent is labeled with a signaling molecule that emits a signal upon excitation.

The method or apparatus of any embodiment of the present disclosure, wherein the thin layer has a uniform thickness that is about equal to or less than 10 μm.

The method or apparatus of any embodiment of the present disclosure, wherein the thin layer has a uniform thickness that is about equal to or less than 2 μm.

The method or apparatus of any embodiment of the present disclosure, wherein the sample is whole blood.

The method or apparatus of any embodiment of the present disclosure, wherein the biomarker is CD3 (cluster of differentiation 3).

The method or apparatus of any embodiment of the present disclosure, wherein the biomarker is CD4 (cluster of differentiation 4).

The method or apparatus of any embodiment of the present disclosure, wherein the biomarker is CD8 (cluster of differentiation 8).

The method or apparatus of any embodiment of the present disclosure, wherein the cells are T cells.

The method or apparatus of any embodiment of the present disclosure, wherein the detecting agent is immobilized on the sample contact area.

The method or apparatus of any embodiment of the present disclosure, wherein the stained cell or bacteria is imaged without washing away the staining solution.

### BRIEF DESCRIPTION OF THE DRAWINGS

A skilled artisan will understand that the drawings, described below, are for illustration purposes only. The drawings are not intended to limit the scope of the present teachings in any way. In some Figures, the drawings are in scale. In the figures that present experimental data points, the lines that connect the data points are for guiding a viewing of the data only and have no other means.

FIG. 1 provides schematic illustrations showing the processes for staining of chlamydia infected cells. Panel (A) shows the conventional method (prior art); panel (B) shows an embodiment of the present invention, where a QMAX device is employed.

FIG. 2 shows exemplary pictures of chlamydia staining with the QMAX device. Panel (A) illustrates the results for the experiments using a 15 second incubation with the antibody to Chlamydia; panel (B) illustrates the results for the experiments using a 30 second incubation with the antibody to Chlamydia.

FIG. 3 shows exemplary pictures of chlamydia staining with the QMAX device without the optional washing step.

FIG. 4 provides a summary of the results from conventional staining and the staining using a QMAX device.

FIG. 5 shows schematic design and results for the staining of chlamydia infected cells. Panel (A) shows the design of the staining process; panel (B) shows an image of immunostaining of Chlamydia infected cells taken with the camera of a smart phone, with one step washing; panel (C) shows an image of immunostaining of Chlamydia infected cells taken with the camera of a smart phone, with no washing.

FIG. 6 shows schematic illustrations of the QMAX device, which is prepared for a one-step sandwich assay for the detection of chlamydia. Panel (A) shows an illustration of the X-plate, to which detection antibodies are attached; panel (B) shows an illustration to demonstrate how the antibody is printed on the X-plate; panel (C) shows an illustration of a PMMA substrate, to which capturing antibodies are attached.

FIG. 7 shows an exemplary flow chart that demonstrates the process to conduct the sandwich assay to detect Chlamydia.

FIG. 8 show an example of the results of sandwich assays that detect chlamydia.

FIG. 9 provides schematic illustrations showing the processes for staining of CD4 expressing cells according to some embodiments of the present invention.

FIG. 10 shows exemplary pictures of CD4 staining with the QMAX device in bright field and fluorescence. The images were captured with inverted microscopy. Panel (A) illustrates the results for the experiments using a QMAX device with a 2 μm gap; panel (B) illustrates the results for the experiments using a QMAX device with a 10 μm gap.

FIG. 11 shows a schematic illustration of the apparatus that is used to capture the images of the sample according to some embodiments of the present invention.

FIG. 12 shows exemplary pictures of CD4 staining with the QMAX device wherein the pictures were captured with the iPhone-laser setup.

FIG. 13 shows an exemplary flow chart that demonstrates the process to conduct the staining assay for CD4 expressing cells.

FIG. 14 shows an illustration of a CROF (Compressed Regulated Open Flow) embodiment. Panel (a) illustrates a first plate and a second plate wherein the first plate has spacers. Panel (b) illustrates depositing a sample on the first plate (shown), or the second plate (not shown), or both (not shown) at an open configuration. Panel (c) illustrates (i) using the two plates to spread the sample (the sample flow between the plates) and reduce the sample thickness, and (ii) using the spacers and the plate to regulate the sample thickness at the closed configuration. The inner surface of each plate may have one or a plurality of binding sites and or storage sites (not shown).

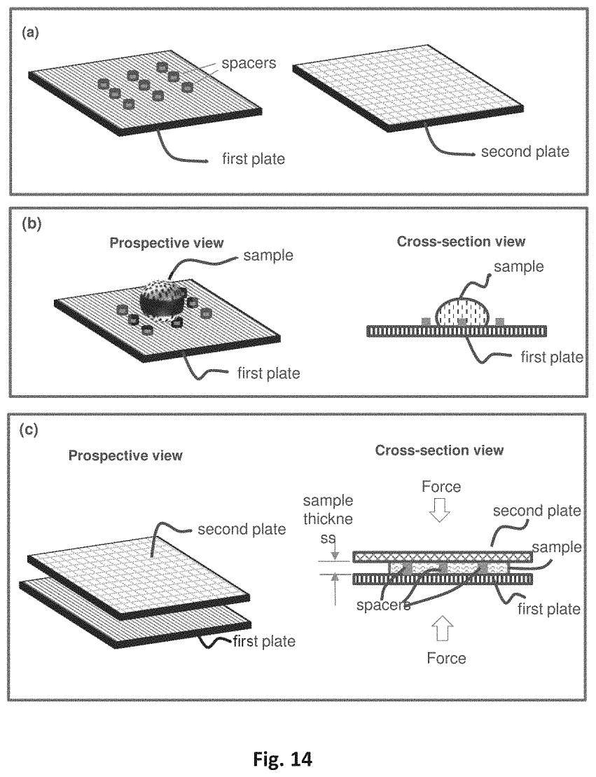

### DETAILED DESCRIPTION OF EXEMPLARY EMBODIMENTS

The following detailed description illustrates some embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It should be noted that the Figures do not intend to show the elements in strict proportion. For clarity purposes, some elements are enlarged when illustrated in the Figures. The dimensions of the elements should be delineated from the descriptions herein provided and incorporated by reference.

In one aspect, the present invention provides detection of bacteria that cause sexually transmitted diseases (STDs). Bacterial STDs are often curable through treatment with antibiotics. Early detection represents one of the regimen and allow patients to follow through on the medication. Because bacterial infection often gives no warning signs or symptoms, early detection using the present device can prevent serious complications caused by the bacterial STDs resulting in irreversible damages of the reproductive organs.

Common sexually transmitted infections caused by bacteria include gonorrhea, syphilis, and chlamydia. In one example embodiment, the present invention provides methods and devices to detect bacteria Chlamydia trachomatis that causes Chlamydia or Lymphogranuloma Venereum. In another example embodiment, the present invention provides methods and device to detect Neisseria gonorrhoeae that causes Gonorrhea. In another example embodiment, the present invention provides methods and devices to detect bacterium Treponema pallidium that causes Syphilis.

In certain embodiments, there is provided a method of detecting a STD-causing bacterium selected from the group consisting of Chlamydia trachomatis, Neisseria gonorrhoeae, and Treponema pallidium.

For merely illustration purposes, the following details of a novel device and method for Chlamydia staining are provided.

### A. Chlamydia Staining

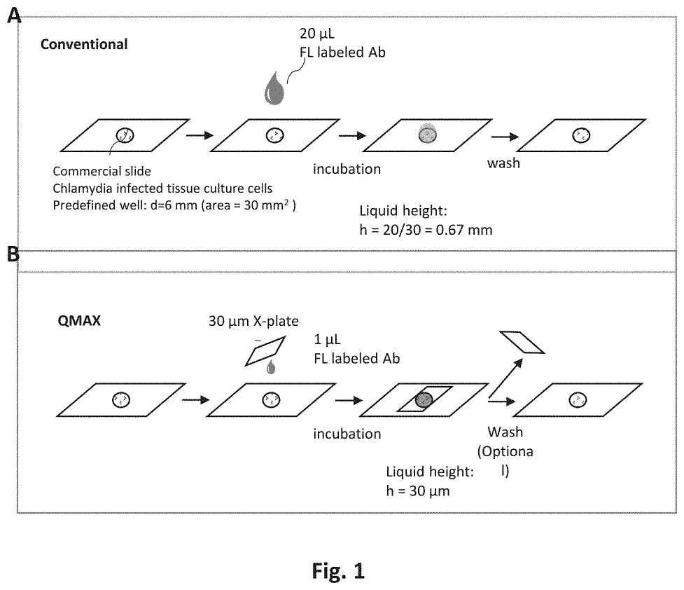

Schematic illustrations are provided showing the processes for staining of chlamydia infected cells (See, FIG. 1). Panel (A) of FIG. 1 shows the conventional method (prior art); panel (B) shows an embodiment of the present invention, where a QMAX device is employed.

In the experiments shown in FIG. 1, mouse anti-chlamydia (Abcam, cat # ab41196) was used for staining and chlamydia antigen control slide (MBL Bion, cat # QCHE-4502) was used as the device to hold the sample.

As shown in panel (A), 20 μL Dylight633 labeled anti-Chlamydia antibody (Abcam) was added to sample slide that carries fixed human tissue cells infected by Chlamydia. The reactants were incubated for a predetermined period of time. The suggested predetermined time period is 30 minutes. Then the slide was washed and the cells were imaged by microscope.

Panel (B) of FIG. 1 shows QMAX staining: 1 uL Dylight633 labeled anti-Chlamydia antibody was added to sample slide that carries fixed human tissue cells infected by Chlamydia. The labeled antibody was then covered by X-plate (30 μm pillar height) for 15 seconds (or longer). In some experiments, as an optional step, the X-plate is removed and the stained slide is washed by dipping into PBST for 3 times and air dried. In some experiments, the slides were not washed. The stained slide and cells were imaged by microscope.

For the conventional method, 20 μl of labeled Ab is the minimum amount for effective staining, compared to 1 μl with the QMAX method.

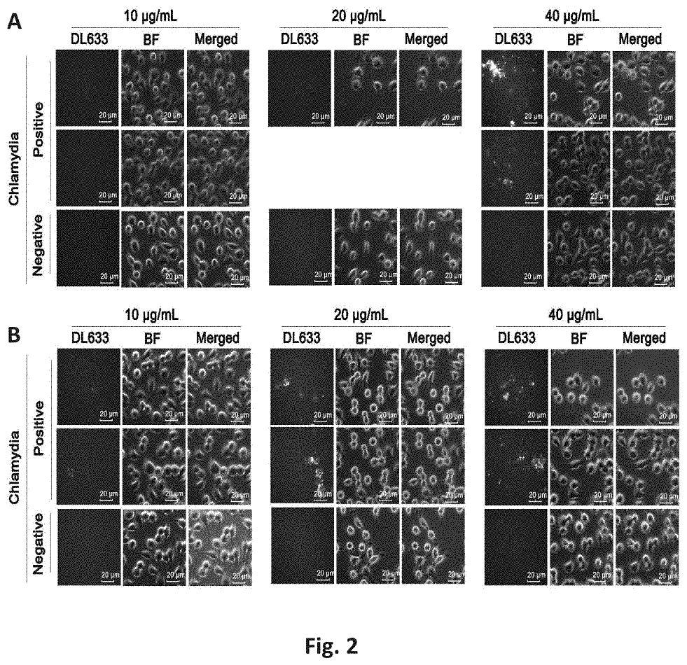

FIG. 2 shows exemplary pictures of chlamydia staining with the QMAX device. Panel (A) illustrates the results for the experiments using a 15 second incubation with the antibody to chlamydia; panel (B) illustrates the results for the experiments using a 30 second incubation with the antibody to chlamydia. DL633 refers to Dylight633 labeled; BF refers to bright field. The “μg/ml” numbers refer to antibody concentration.

In the experiments shown in FIG. 2, slides with fixed human tissue cells infected by chlamydia were incubated with 1 μL of DyLight633 labeled anti-chlamydia antibody with different concentration for 15 seconds (panel A) or 30 seconds (panel B). X-plate with 30 μm pillar height was used in the QMAX immunostaining. The slides were then washed by PBST and images were taken by microscope. BF: bright field. DL633: DyLight633 fluorescence.

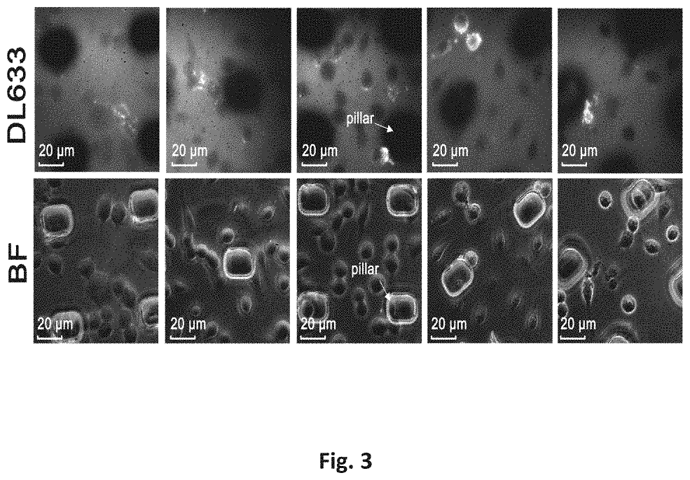

FIG. 3 shows exemplary pictures of chlamydia staining with the QMAX device without the optional washing step. In the experiments shown in FIG. 3, slides with fixed human tissue cells infected by chlamydia were incubated with 1 μL of DyLight633 labeled anti-chlamydia antibody with 40 μg/mL for 2 min. X-plate with 30 μm pillar height was used in the QMAX immunostaining. Images were taken by microscope without washing. BF: bright field. DL633: DyLight633 fluorescence.]

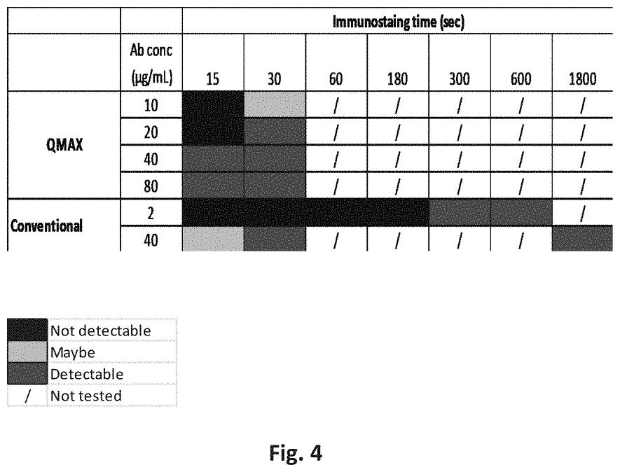

FIG. 4 provides a summary of the results from conventional staining and the staining using a QMAX device. It is noted that QMAX immunostaining of Chlamydia can be detected after 15 s (40 μg/mL).

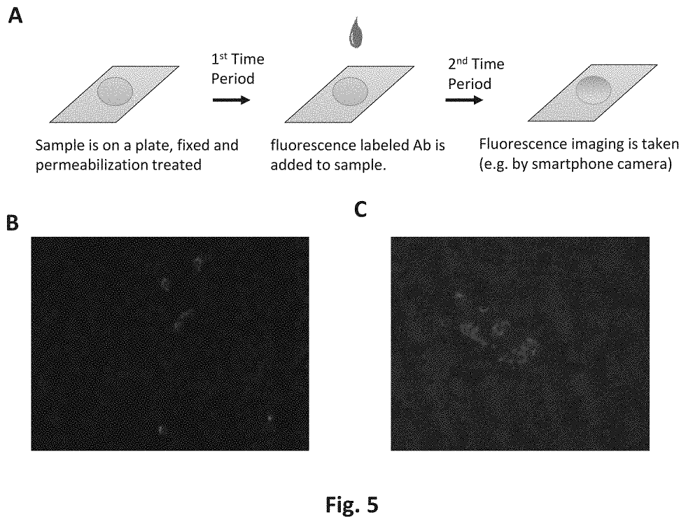

FIG. 5 shows schematic design and results for the staining of chlamydia infected cells. Panel (A) shows the design of the staining process. In some embodiments, the sample is placed on a plate. In certain embodiments, the sample includes cells that are suspected to be infected by chlamydia. In certain embodiments, the sample is immobilized on the plate. In certain embodiments, the sample is fixed and/or permeabilized. In certain embodiments, the sample is not immobilized.

Panel (B) shows an image of immunostaining of Chlamydia infected cells taken with the camera of a smart phone, with one step washing. Panel (C) shows an image of immunostaining of Chlamydia infected cells taken with the camera of a smart phone, with no washing.

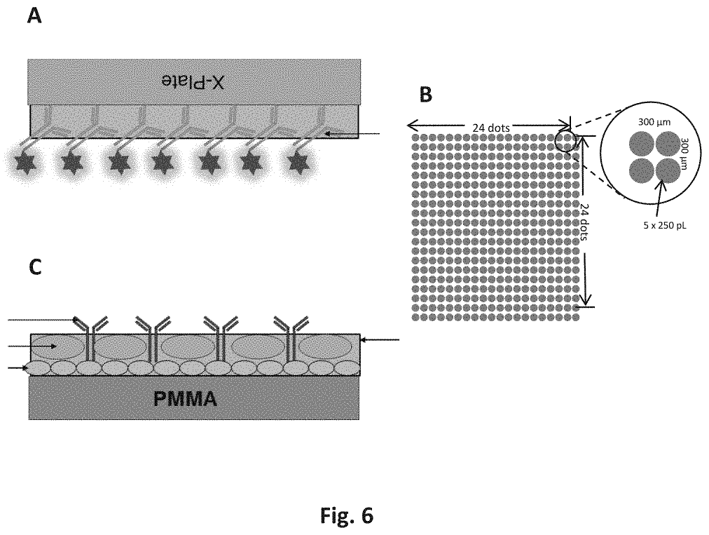

FIG. 6 shows schematic illustrations of the QMAX device, which is prepared for a one-step sandwich assay for the detection of chlamydia. Panel (A) shows an illustration of the X-plate, to which detection antibodies are attached; panel (B) shows an illustration to demonstrate how the antibody is printed on the X-plate; panel (C) shows an illustration of a PMMA substrate, to which capturing antibodies are attached.

As shown in panel (B) of FIG. 6, Nanoprint DyLight633-anti-chlamydia detection antibody (100 μg/mL) in PBST and 1:100 commercial protein stabilizer on pre-cut X-Plates, dry at room temperature.

Setup: 8×2 arrays, 24×24 dots per array, 5×250 μL per dot. Array size: 7.2 mm×7.2 mm. Gap between dots: 300 μm.

PMMA as binding site: 100 μL Protein A 20 μg/mL in PBS coat substrate overnight/Wash 3× with PBST; 100 μL Capture Ab 20 μg/mL in PBS coat substrate for 3 h/Wash 3× with PBST; 100 μL Blocking the substrate with 4% BSA in PBS for 2 h/Wash 3× with PBST; incubate 100 μL StabilCoat stabilizer for 1 h/pipette excessive/Dry at RT. It is noted that 6.5×6.5 mm FlexWell was used on PMMA.

The antibodies used in the experiments shown in the sandwich assays are listed in the Table below.

TABLE 1

| Antibody | Vendor | Cat # |
|---|---|---|
| Mouse anti-chlamydia | Fitzgerald | 10-C13A |
| (Capture antibody) | | |
| Mouse anti-chlamydia | Fitzgerald | 10-C13B |
| (Capture antibody) | | |

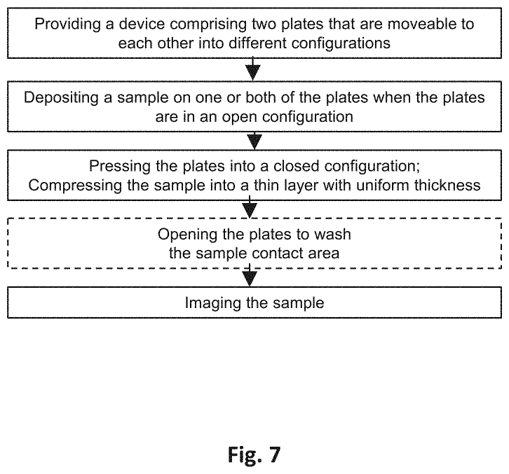

FIG. 7 shows an exemplary flow chart that demonstrates the process to conduct the sandwich assay to detect chlamydia. In some experiments, a series samples, each with a volume of 0.8 μL, containing different concentrations of chlamydia analyte were deposited on coated substrate at different locations. X-plate nanoprinted with the detection antibody was pressed on top of the liquid by hand. The reactants were incubated for 2 min. In some experiments, one step washing was performed: peel off the X-plate; wash the substrate plate binding site in PBST for 1 min and then water briefly. The results were measure by Raman microscope.

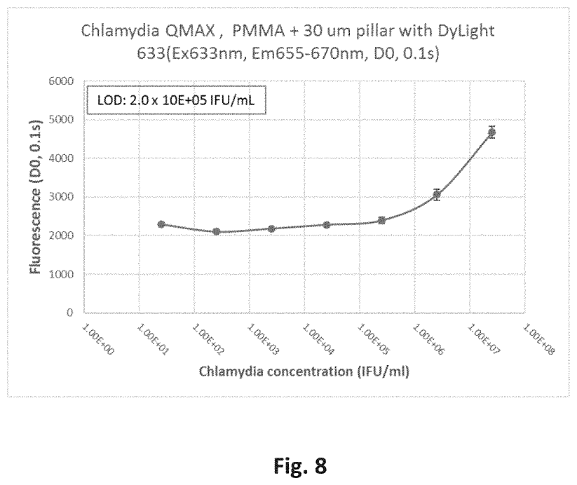

FIG. 8 show an example of the results of sandwich assays that detect chlamydia. FIG. 8 provides a standard curve of chlamydia QMAX sandwich immunoassay. 1 μL of purified chlamydia elementary bodies (EB) were used in the QMAX assay. Limit of detection (LOD): 2×10E+05 IFU/mL, or 200 IFU per test.

In one embodiment, the present invention provides a one step method of staining assay for bacteria that causes chlamydia, gonorrhea, or syphilis.

In a preferred embodiment, the present invention provides a method for detecting chlamydia in a sample, comprising:
(a) obtaining a first plate comprising, on its inner surface, a sample contact area that is configured to contact a sample;
(b) depositing the sample in the sample contact area, wherein the sample contains or is suspected to comprise cells infected with chlamydia, gonorrhea or syphilis; and
(c) depositing a chlamydia, gonorrhea or syphilis staining medium on the sample, wherein the staining medium comprises a bacteria-binding antibody, and the staining medium and the sample form a mixture;
(d) covering the mixture of the sample and the staining medium with a second plate,
(e) pressing the first plate and the second plate so that at least part of the mixture is compressed into a thin layer;
(f) incubating for a period of time that is about 60 seconds or less; and
(g) detecting a chlamydia, gonorrhea or syphilis-related signal from the mixture.

In certain embodiments, the predetermined period of time that is about 30 seconds or less. In certain embodiments, the period of time that is about 15 seconds or less.

In certain embodiments, the chlamydia, gonorrhea or syphilis antibody is fluorescently labeled.

In certain embodiments, the thin layer has a uniform thickness that is less than 100 μm. In certain embodiments, the thin layer has a uniform thickness that is less than 50 μm. In certain embodiments, the thin layer has a uniform thickness that is about 30 μm or less.

In certain embodiments, the chlamydia-related signal is detected by imaging the sample.

In one embodiment, the present invention provides a one step sandwich assay for testing chlamydia, gonorrhea, or syphilis. In a preferred embodiment, the present invention provides a method for detecting chlamydia, gonorrhea or syphilis in a sample, comprising:
(a) providing a first plate comprising, on its inner surface, a sample contact area that has a binding site, wherein the binding site comprises an immobilized capture antibody that binds to chlamydia in a sample that contains or is suspected to contain chlamydia, gonorrhea or syphilis;
(b) providing a second plate comprising, on its inner surface, a sample contact area that has a storage site, wherein the storage site comprises a detection antibody that is capable of, upon contacting the sample, diffusing in the sample, and wherein the capture antibody and detection antibody bind to different sites in the chlamydia to form a capture antibody-bacteria-detection antibody sandwich;
(c) depositing the sample on one or both of the sample contact areas of the plates;
(d) after (c), bringing the two plates to a close configuration, wherein, in the closed configuration, at least part of the sample deposited in (c) is confined between the sample contact areas of the two plates, and has an average thickness in the range of 0.01 μm to 200 μm; and
(e) detecting a signal related to chlamydia captured by the capture antibody.

In certain embodiments, the sample is from a human subject.

In certain embodiments, the capturing site further comprises a protein stabilizer.

In certain embodiments, the storage site further comprises a protein stabilizer.

In certain embodiments, the detection antibody comprises a fluorescent label.

In certain embodiments, the sample between the two plates has a uniform thickness in the range of 0.5 μm to 50 μm. In certain embodiments, the sample between the two plates has a uniform thickness in the range of 1 μm to 35 μm.

In certain embodiments, the method further comprising determining the presence or absence of chlamydia, gonorrhea or syphilis.

In certain embodiments, the overall time for steps (a)-(e) is less than 10 minutes. In certain embodiments, the overall time for steps (e)-(e) is less than 3 minutes. In certain embodiments, the overall time for steps (a)-(e) is less than 2 minutes.

Antibody against chlamydia, gonorrohea or syphilis can be obtained via commercial sources. Exemplary anti-syphilis antibody includes anti-TP17 (Cat # R8A201), or anti-Tp15 (Cat # R8A101) or anti-Tp47 (Cat # R8A403) from Meridianlife. Exemplary anti-chlamydia includes Abcam (Cat # Ab41196). Exemplary anti-gonorrohea Abcam (Cat # Ab62964; Ab19962).

### Additional Features

In certain embodiments, one or both of the sample contact areas comprise spacers, wherein the spacers regulate the spacing between the sample contact areas of the plates when the plates are in the closed configuration.

In certain embodiments, the first plate comprises a plurality of binding sites and the second plate comprises a plurality of corresponding storage sites, wherein each biding site faces a corresponding storage site when the plates are in the closed configuration.

In certain embodiments, the detection antibody is dried on the storage site.

In certain embodiments, the capture antibody at the binding site are on an amplification surface that amplifies an optical signal of the analytes or the captured detection agents in any prior embodiments.

In certain embodiments, the capture agents at the binding site are on an amplification surface that amplifies an optical signal of the analytes or the captured detection agents in any prior embodiments, wherein the amplification is proximity-dependent in that the amplification significantly reduced as the distance between the capture agents and the analytes or the detection agents increases.

In certain embodiments, the detection of the signal is electrical, optical, or both. (including but not limited to Fluorescence, SPR, etc.).

In one aspect, the present invention provides staining of immune cells such as T-immune cells. In particular, the present invention provides a novel device and method for detecting and quantifying CD3+, CD4+ and CD8+ cells.

Cluster of differentiation 3 (CD3) is a multimeric protein complex, known historically as the T3 complex, and is composed of four distinct polypeptide chains; epsilon (ε), gamma (γ), delta (δ) and zeta (ζ), that assemble and function as three pairs of dimers (εγ, εδ, ζζ). The CD3 complex serves as a T cell co-receptor that associates noncovalently with the T cell receptor (TCR). The CD3 protein complex is a defining feature of the T cell lineage, therefore anti-CD3 antibodies can be used effectively as T cell markers.

Within the CD3 cell population, CD4 and CD8 represent the two T-cell subsets have different TCR gene rearrangement patterns, tissue distributions and mechanisms of antigen recognition. CD4+ T cells express the CD4 glycoprotein on their surfaces. CD4 Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. CD8⁺ T cells since they express the CD8 glycoprotein at their surfaces. These cells recognize their targets by binding to antigen associated with MHC class I molecules, which are present on the surface of all nucleated cells.

In certain embodiments, there is provided a method of detecting a T-cell selected from the group consisting of CD4 T-cell, CD3 T-cell and CD8 T-cell.

For merely illustration purposes, the following details is provided for a novel device and method for CD4 staining for the T immune cells.

### B. Quantifying CD4 Expressing Cells

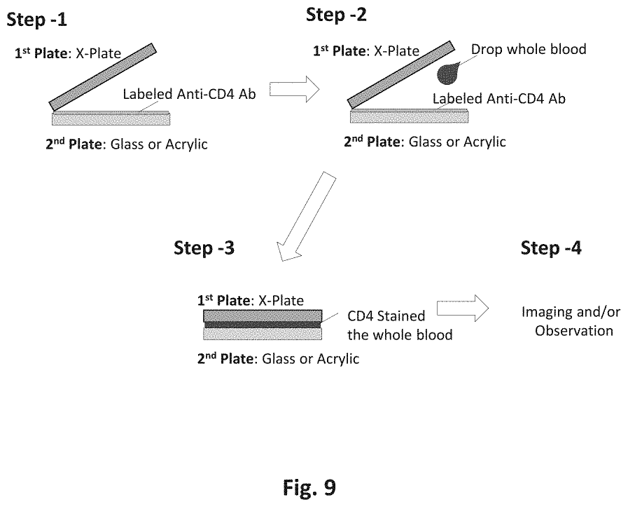

Schematic illustrations are provided (See, FIG. 9) showing the processes for staining of CD4 expressing cells according to some embodiments of the present invention.

As shown in FIG. 9, in some embodiments, a first plate (termed “X-plate”) and a second plate (e.g. made from glass or acrylic) were obtained, wherein the first plate and the second plate are moveable relative to each other. In certain embodiments, the first plate and the second plate are not connected. In certain embodiments, the first plate and the second plate are connected by a turning structure (e.g. a hinge). Each of the plates have two surfaces: one inner surface and one outer surface, wherein the inner surfaces face each other when the plates are pressed against each other. On the inner surfaces, each plate comprises a sample contact area for contacting a liquid sample.

In some embodiments, a detecting agent (e.g. a labeled anti-CD4 antibody) is immobilized on the sample contact area of one or both of the plates. In certain embodiments, the detecting agent comprises an anti-CD4 antibody. In certain embodiments, the detecting agent is labeled with a fluorophore. In certain embodiments, as shown in FIG. 9, the anti-CD4 antibody is labeled with Alex 647.

In step 2, when the plates are in an open configuration, in which the plates are separated apart, a liquid sample is deposited on the sample contact area of one or both of the plates. In certain embodiments, as shown in FIG. 9, the sample is whole blood.

In step 3, the plates are pressed against each other into a closed configuration. In certain embodiments, the pressing is conducted with human hand. In the closed configuration, the plates are pressed against each other with a gap between them, and the sample is compressed into a thin layer. In certain embodiments, the thin layer has a uniform thickness. In certain embodiments, one of both of the plates comprise spacers that are fixed in one or both of the sample contact areas. When the plates are pressed into the closed configuration, the spacers regulate the thickness of the sample layer. In certain embodiments, the spacers have a pillar shape.

In step 4, the sample layer is imaged and the number of CD4 expressing cells are quantified.

In the experiments shown in FIG. 9, the QMAX device has two plates. The first plate was a X-Plate with 2 μm or 10 μm pillar height, 30×40 um pillar size, 80 um inter spacing distance, and is made of 175 um thick PMMA. The second plate was 1 mm thick glass or acrylic. The Anti-CD 4 antibody with label Alex 647 was on positioned on the second plate in either liquid form or dry form. In its liquid form, the anti-CD4 antibody is 5 to 50 μg/mL with a volume of 0.5 to 1 μL. The anti-CD4 antibody was printed into an array of 300 um period and dried, with a surface concentration of 1 to 100 ng/cm² after drying.

In the experiments shown in FIG. 9, for step 2, the sample is fresh whole blood with a volume of ˜1 μL.

In the experiment shown in FIG. 9, for step 3, after the plates are pressed against each other, the sample layer is incubated with the detecting agent for about 60 seconds.

In the experiment shown in FIG. 9, for step 4, the stained whole blood sample layer was imaged either with laboratory microscopy or with an mobile device-adaptor system.

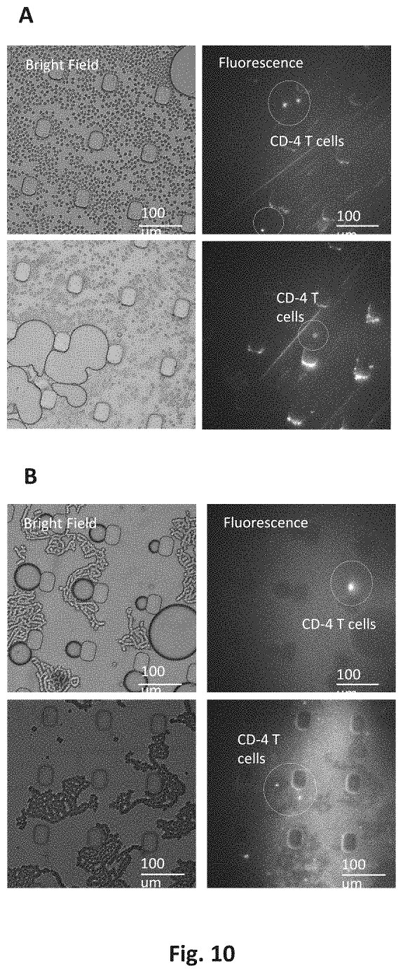

FIG. 10 shows exemplary pictures of CD4 staining with the QMAX device in bright field and fluorescence. The images were captured with inverted microscopy. Panel (A) illustrates the results for the experiments using a QMAX device with a 2 μm gap; panel (B) illustrates the results for the experiments using a QMAX device with a 10 μm gap.

As shown in FIG. 10, for panel (A), the bright field photo illustrates both red blood cells and white blood cells; the fluorescence photo shows clear fluorescence of stained CD4 T cells. As shown in FIG. 10, for panel (B), the bright field photo illustrates aggregated red blood cells and white blood cells; the fluorescence photo shows clear fluorescence of stained CD4 T cells.

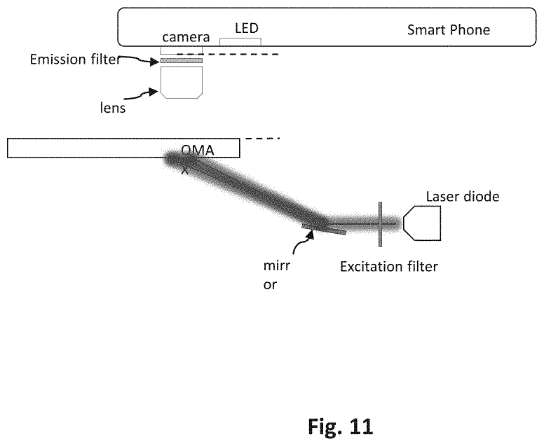

FIG. 10 shows a schematic illustration of the apparatus that is used to capture the images of the sample according to some embodiments of the present invention. With iPhone as an example, FIG. 11 shows the iPhone/reader setup with laser diode as a light source. The laser diode has 638 nm central wavelength with 10 to 20 mW power. The excitation filter before the light source is 650 nm short pass. The light is reflected by an aluminum mirror onto the back of QMAX device with a typical illumination area of 1 mm×4 mm. The observation system is at the front of QMAX device with an iPhone adding an emission filter and lens. The emission filter is 670 nm long pass. The lens has focus distance around 4 mm and N.A. of 0.2.

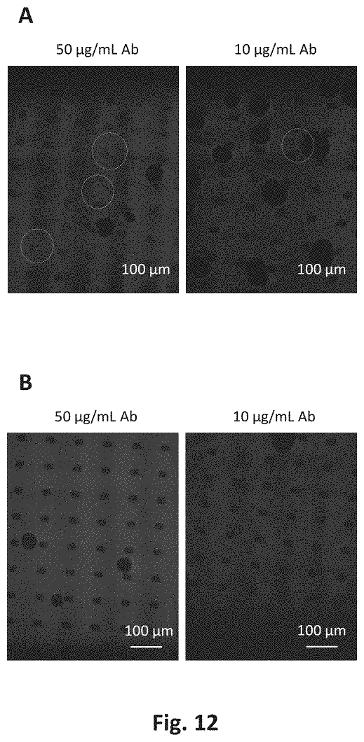

FIG. 12 shows exemplary pictures of CD4 staining with the QMAX device wherein the pictures were captured with the iPhone-laser setup shown in FIG. 11. Fluorescence photo of CD4 stained whole blood in 2 μm thick QMAX card under phone/reader system. The left photo is using relative high antibody concentration of 50 μg/mL and right photo is using antibody concentration of 10 μg/mL. The fluorescence photo shows clear fluorescence of stained CD4 T cells. (b) Fluorescence photo of CD4 stained whole blood in 10 μm thick QMAX card under phone/reader system. The left photo is using relative high antibody concentration of 50 μg/L and right photo is using antibody concentration of 10 μg/mL. CD4 T cells is not observed under 10 μm QMAX, which might due to the iPhone reader's lower sensitivity and dynamic range compared with inverted microscopy system.

The number of CD4 expressing T cells counted with the QMAX device and the iPhone-laser setup as shown in FIG. 11 are listed in the Table below.

TABLE 2
Back- Calculated CD4 T Cells Concentration
from QMAX/MOST (Mobile Self Test).

| | QMAX/MOST value | Control Value |
|---|---|---|
| CD4 T Cells | 900/μL | 500-1600/μL |

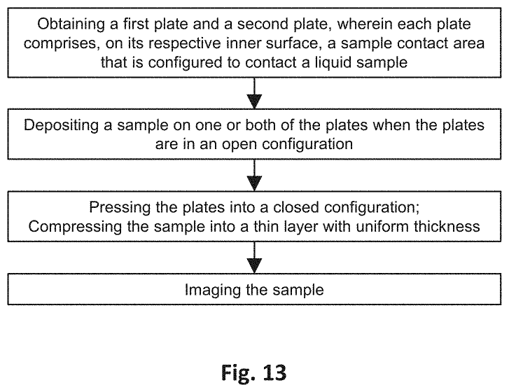

FIG. 13 shows an exemplary flow chart that demonstrates the process to conduct the staining assay for CD4 expressing cells.

Antibodies to CD3, CD4 or CD8 can be conveniently obtained via commercial source. Exemplary CD4 antibody includes Fitzgerald Industries international (Cat #10R-CD4KHUP), or Thermo Fisher (Cat # MA1-81407). Exemplary CD8 antibody includes Thermo Fisher (Cat # MHCD0800), or Fitzgerald Industries international (Cat #10R-1881). Exemplary CD3 antibody includes Thermo Fisher (Cat # MHCD0300) and Fitzgerald Industries international (Cat # UCH-T1).

In some embodiments, the staining comprises the following steps:
(a) obtaining a first plate and a second plate, wherein each plate comprises, on its respective inner surface, a sample contact area that is configured to contact a liquid sample,
   wherein a detecting agent is positioned on the sample contact area of one or both of the plates, and
   wherein the detecting agent is configured to specifically bind to the biomarker,
(b) depositing the sample in the sample contact area, wherein the sample comprises cells that express the biomarker;
(c) pressing the first plate and the second plate to compress the sample into a thin layer, which is at least partly confined by the two sample contact areas that face each other;
(d) incubating for a predetermined period of time that is about 60 seconds or less; and
(e) quantifying the cells expressing the biomarker by imaging the sample layer and counting the cells expressing the biomarker.
   One Step Staining Assay for CD3, CD4 or CD8 T Cells in Whole Blood without Wash

In certain embodiments, the present invention provides a method for quantifying cells that express a biomarker (CD3, CD4 or CD8) in a sample, comprising:
(a) obtaining a sample holder that is configured to hold a liquid sample that contains an analyte, wherein a detecting agent is positioned in the sample holder and is configured to specifically bind to the biomarker;
(b) depositing the sample in the sample contact area, wherein the sample comprises cells that express the biomarker; and the sample is in contact with the detecting agent in the sample holder;
(c) adjusting the sample holder to compress the sample into a thin layer,
(d) incubating for a predetermined period of time; and
(e) quantifying the cells expressing the biomarker by imaging the sample layer and counting the cells expressing the biomarker.

In certain embodiments, the present invention provides a method for quantifying cells that express a biomarker in a sample, comprising:
(a) obtaining a first plate and a second plate, wherein each plate comprises, on its respective inner surface, a sample contact area that is configured to contact a liquid sample,
   wherein a detecting agent is positioned on the sample contact area of one or both of the plates, and wherein the detecting agent is configured to specifically bind to the biomarker,
(b) depositing the sample in the sample contact area, wherein the sample comprises cells that express the biomarker (CD3, CD4 or CD8);
(c) pressing the first plate and the second plate to compress the sample into a thin layer, which is at least partly confined by the two sample contact areas that face each other;
(d) incubating for a predetermined period of time that is about 60 seconds or less; and
(e) quantifying the cells expressing the biomarker by imaging the sample layer and counting the cells expressing the biomarker.

In certain embodiments, the present invention provides a method for quantifying cells that express CD3, CD4, or CD8 (cluster of differentiations 3, 4 or 8)) in a blood sample, comprising:
(a) obtaining a first plate and a second plate, wherein each plate comprises, on its respective inner surface, a sample contact area that is configured to contact a blood sample,
   wherein a detecting agent is positioned on the sample contact area of one or both of the plates, and
   wherein the detecting agent is configured to specifically bind to CD3, CD4 or CD8,
(b) depositing the blood sample in the sample contact area, wherein the blood sample comprises cells that express CD3, CD4 or CD8;
(c) pressing the first plate and the second plate to compress the blood sample into a thin layer, which is at least partly confined by the two sample contact areas that face each other;
(d) incubating for a predetermined period of time that is about 60 seconds or less; and quantifying the CD3, CD4 or CD8 expressing cells by imaging the sample layer and counting the cells expressing CD3, CD4, or CD8.

In certain embodiments, the present invention provides an apparatus for quantifying cells that express a biomarker in a sample, comprising:

a sample holder that is configured to hold a liquid sample that contains cells that express a biomarker, wherein a detecting agent is positioned in the sample holder and is configured to specifically bind to the biomarker; and

an adaptor that is configured to accommodate the sample holder and be attachable to a mobile device, wherein:

i. the mobile device comprises an imager,

ii. the adaptor is configured to position the sample in a field of view (FOV) of the imager when the adaptor is attached to the mobile device, and

iii. the imager is configured to capture images of the sample, thereby detecting/measuring a signal that is generated by the binding of the biomarker with the detecting agent after the sample is incubated with the detecting agent for a predetermined period of time that is about 60 seconds or less.

Preferably, the predetermined period of time that is about 30 seconds or less.

Preferably, the present assay does not involve the sample contact areas to be washed after step (d).

Preferably, the detecting agent is an antibody. Preferably, the antibody is labeled with a fluorophore.

Preferably, the detecting agent is labeled with signaling molecule that emits a signal upon excitation.

Preferably, the thin layer has a uniform thickness that is about equal to or less than 10 μm. More preferably, the thin layer has a uniform thickness that is about equal to or less than 2 μm.

Preferably, the sample is whole blood.

Preferably, the biomarker is CD3, CD4 or CD8 (cluster of differentiation 3, 4, or 8). More preferably, the cells are T cells.

Preferably, the detecting agent is immobilized on the sample contact area.

Device and Assay with High Uniformity

Flat Top of Pillar Spacers

In certain embodiments of the present invention, the spacers are pillars that have a flat top and a foot fixed on one plate, where the flat top has a smoothness with a small surface variation, and the variation is less than 5, 10 nm, 20 nm, 30 nm, 50 nm, 100 nm, 200 nm, 300 nm, 400 nm, 500 nm, 600 nm, 700 nm, 800 nm, 1000 nm, or in a range between any two of the values. A preferred flat pillar top smoothness is that surface variation of 50 nm or less.

Furthermore, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

Sidewall Angle of Pillar Spacers

In certain embodiments of the present invention, the spacers are pillars that have a sidewall angle. In some embodiments, the sidewall angle is less than 5 degree (measured from the normal of a surface), 10 degree, 20 degree, 30 degree, 40 degree, 50 degree, 70 degree, or in a range between any two of the values. In a preferred embodiment, the sidewall angle is less than 5 degree, 10 degree, or 20 degree.

Formation of Uniform Thin Fluidic Layer by an Imprecise Force Pressing

In certain embodiment of the present invention, a uniform thin fluidic sample layer is formed by using a pressing with an imprecise force. The term “imprecise pressing force” without adding the details and then adding a definition for imprecise pressing force. As used herein, the term “imprecise” in the context of a force (e.g. “imprecise pressing force”) refers to a force that
(a) has a magnitude that is not precisely known or precisely predictable at the time the force is applied;
(b) has a pressure in the range of 0.01 kg/cm² (centimeter square) to 100 kg/cm²,
(c) varies in magnitude from one application of the force to the next; and
(d) the imprecision (i.e. the variation) of the force in (a) and (c) is at least 20% of the total force that actually is applied.

An imprecise force can be applied by human hand, for example, e.g., by pinching an object together between a thumb and index finger, or by pinching and rubbing an object together between a thumb and index finger.

In some embodiments, the imprecise force by the hand pressing has a pressure of 0.01 kg/cm², 0.1 kg/cm², 0.5 kg/cm², 1 kg/cm², 2 kg/cm², kg/cm², 5 kg/cm², 10 kg/cm², 20 kg/cm², 30 kg/cm², 40 kg/cm², 50 kg/cm², 60 kg/cm², 100 kg/cm², 150 kg/cm², 200 kg/cm², or a range between any two of the values; and a preferred range of 0.1 kg/cm² to 0.5 kg/cm², 0.5 kg/cm² to 1 kg/cm², 1 kg/cm² to 5 kg/cm², 5 kg/cm² to 10 kg/cm² (Pressure).

Spacer Filling Factor.

The term “spacer filling factor” or “filling factor” refers to the ratio of the spacer contact area to the total plate area”, wherein the spacer contact area refers, at a closed configuration, the contact area that the spacer's top surface contacts to the inner surface of a plate, and the total plate area refers the total area of the inner surface of the plate that the flat top of the spacers contact. Since there are two plates and each spacer has two contact surfaces each contacting one plate, the filling fact is the filling factor of the smallest.

For example, if the spacers are pillars with a flat top of a square shape (10 um×10 um), a nearly uniform cross-section and 2 μm tall, and the spacers are periodic with a period of 100 μm, then the filing factor of the spacer is 1%. If in the above example, the foot of the pillar spacer is a square shape of 15 um×15 um, then the filling factor is still 1% by the definition.

IDS∧4/hE

In one embodiment, there is provided a device for forming a thin fluidic sample layer with a uniform predetermined thickness by pressing, comprising:

a first plate, a second plate, and spacers, wherein:
i. the plates are movable relative to each other into different configurations;
ii. one or both plates are flexible;
iii. each of the plates comprises an inner surface that has a sample contact area for contacting a fluidic sample;
iv. each of the plates comprises, on its respective outer surface, a force area for applying a pressing force that forces the plates together;
v. one or both of the plates comprise the spacers that are permanently fixed on the inner surface of a respective plate;
vi. the spacers have a predetermined substantially uniform height that is equal to or less than 200 microns, and a predetermined fixed inter-spacer-distance;
vii. the fourth power of the inter-spacer-distance (ISD) divided by the thickness (h) and the Young's modulus (E) of the flexible plate (ISD⁴/(hE)) is 5×10⁶ μm/GPa or less; and
viii. at least one of the spacers is inside the sample contact area;

wherein one of the configurations is an open configuration, in which: the two plates are partially or completely separated apart, the spacing between the plates is not regulated by the spacers, and the sample is deposited on one or both of the plates;

wherein another of the configurations is a closed configuration which is configured after the sample is deposited in the open configuration and the plates are forced to the closed configuration by applying the pressing force on the force area; and in the closed configuration:

at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In one embodiment, there is provided a method of forming a thin fluidic sample layer with a uniform predetermined thickness by pressing, comprising the steps of:
(a) obtaining the device described in the previous embodiment;
(b) depositing a fluidic sample on one or both of the plates; when the plates are configured in an open configuration, wherein the open configuration is a configuration in which the two plates are partially or completely separated apart and the spacing between the plates is not regulated by the spacers;
(c) after (b), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In one embodiment, there is provided a device for analyzing a fluidic sample, comprising:

a first plate, a second plate, and spacers, wherein:
i. the plates are movable relative to each other into different configurations;
ii. one or both plates are flexible;
iii. each of the plates has, on its respective inner surface, a sample contact area for contacting a fluidic sample,
iv. one or both of the plates comprise the spacers and the spacers are fixed on the inner surface of a respective plate;
v. the spacers have a predetermined substantially uniform height that is equal to or less than 200 microns, and the inter-spacer-distance is predetermined;
vi. the Young's modulus of the spacers multiplied by the filling factor of the spacers is at least 2 MPa; and
vii. at least one of the spacers is inside the sample contact area; and

wherein one of the configurations is an open configuration, in which: the two plates are partially or completely separated apart, the spacing between the plates is not regulated by the spacers, and the sample is deposited on one or both of the plates; and

wherein another of the configurations is a closed configuration which is configured after the sample is deposited in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In one embodiment, there is provided a method of forming a thin fluidic sample layer with a uniform predetermined thickness by pressing, comprising the steps of:
(a) obtaining the device described in the previous embodiment;
(b) depositing a fluidic sample on one or both of the plates; when the plates are configured in an open configuration, wherein the open configuration is a configuration in which the two plates are partially or completely separated apart and the spacing between the plates is not regulated by the spacers;
(c) after (b), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In one embodiment, there is provided a device for analyzing a fluidic sample, comprising: a first plate and a second plate, wherein:
i. the plates are movable relative to each other into different configurations;
ii. one or both plates are flexible;
iii. each of the plates has, on its respective surface, a sample contact area for contacting a sample that contains an analyte,
iv. one or both of the plates comprise spacers that are permanently fixed to a plate within a sample contact area, wherein the spacers have a predetermined substantially uniform height and a predetermined fixed inter-spacer distance that is at least about 2 times larger than the size of the analyte, up to 200 μm, and wherein at least one of the spacers is inside the sample contact area;

wherein one of the configurations is an open configuration, in which: the two plates are separated apart, the spacing between the plates is not regulated by the spacers, and the sample is deposited on one or both of the plates; and

wherein another of the configurations is a closed configuration which is configured after the sample deposition in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In one embodiment, there is provided a method of forming a thin fluidic sample layer with a uniform predetermined thickness by pressing, comprising the steps of:
(a) obtaining the device described in the previous embodiment;
(b) depositing a fluidic sample on one or both of the plates; when the plates are configured in an open configuration, wherein the open configuration is a configuration in which the two plates are partially or completely separated apart and the spacing between the plates is not regulated by the spacers;
(c) after (b), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In one embodiment, there is provided a device for forming a thin fluidic sample layer with
a uniform predetermined thickness by pressing, comprising:
a first plate, a second plate, and spacers, wherein:
   i. the plates are movable relative to each other into different configurations;
   ii. one or both plates are flexible;
   iii. each of the plates comprises, on its respective inner surface, a sample contact area for contacting and/or compressing a fluidic sample;
   iv. each of the plates comprises, on its respective outer surface, an area for applying a force that forces the plates together;
   v. one or both of the plates comprise the spacers that are permanently fixed on the inner surface of a respective plate;
   vi. the spacers have a predetermined substantially uniform height that is equal to or less than 200 microns, a predetermined width, and a predetermined fixed inter-spacer-distance;
   vii. a ratio of the inter-spacer-distance to the spacer width is 1.5 or larger; and
   viii. at least one of the spacers is inside the sample contact area;

wherein one of the configurations is an open configuration, in which: the two plates are partially or completely separated apart, the spacing between the plates is not regulated by the spacers, and the sample is deposited on one or both of the plates;

wherein another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In one embodiment, there is provided a method of forming a thin fluidic sample layer with a uniform predetermined thickness by pressing with an imprecise pressing force, comprising the steps of:
(a) obtaining the device described in the previous embodiment;
(b) obtaining a fluidic sample;
(c) depositing the sample on one or both of the plates; when the plates are configured in an open configuration, wherein the open configuration is a configuration in which the two plates are partially or completely separated apart and the spacing between the plates is not regulated by the spacers;
(d) after (c), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

Preferably, the spacers have a shape of pillar with a foot fixed on one of the plate and a flat top surface for contacting the other plate. Preferably, the spacers have a shape of pillar with a foot fixed on one of the plate, a flat top surface for contacting the other plate, substantially uniform cross-section.

Preferably, the spacers have a shape of pillar with a foot fixed on one of the plate and a flat top surface for contacting the other plate, wherein the flat top surface of the pillars has a variation in less than 10 nm. More preferably, the spacers have a shape of pillar with a foot fixed on one of the plate and a flat top surface for contacting the other plate, wherein the flat top surface of the pillars has a variation in less than 50 nm. More preferably, the devices or methods of any prior embodiment, wherein the spacers have a shape of pillar with a foot fixed on one of the plate and a flat top surface for contacting the other plate, wherein the flat top surface of the pillars has a variation in less than 10 nm, 20 nm, 30 nm, 100 nm, 200 nm, or in a range of any two of the values.

Preferably, the Young's modulus of the spacers multiplied by the filling factor of the spacers is at least 2 MPa. More preferably, the Young's modulus of the spacers multiplied by the filling factor of the spacers is at least 20 MPa.

Preferably, the sample comprises an analyte and the predetermined constant inter-spacer distance is at least about 2 times larger than the size of the analyte, up to 200 μm. More preferably, the sample comprise an analyte and the predetermined constant inter-spacer distance is at least about 2 times larger than the size of the analyte, up to 200 μm, and the Young's modulus of the spacers multiplied by the filling factor of the spacers is at least 2 MPa.

Preferably, there is provided a fourth power of the inter-spacer-distance (IDS) divided by the thickness (h) and the Young's modulus (E) of the flexible plate (ISD∧4/(hE)) is 5×10⁶ μm³/GPa or less. More preferably, there is provided a fourth power of the inter-spacer-distance (IDS) divided by the thickness (h) and the Young's modulus (E) of the flexible plate (ISD∧4/(hE)) is 1×10⁶ μm³/GPa or less. More preferably, there is provided a fourth power of the inter-spacer-distance (IDS) divided by the thickness (h) and the Young's modulus (E) of the flexible plate (ISD∧4/(hE)) is 5×10⁵ μm³/GPa or less.

Preferably, the Young's modulus of the spacers multiplied by the filling factor of the spacers is at least 2 MPa, and a fourth power of the inter-spacer-distance (IDS) divided by the thickness (h) and the Young's modulus (E) of the flexible plate (ISD∧4/(hE)) is 1×10⁵ μm³/GPa or less. More preferably, the Young's modulus of the spacers multiplied by the filling factor of the spacers is at least 2 MPa, and a fourth power of the inter-spacer-distance (IDS) divided by the thickness (h) and the Young's modulus (E) of the flexible plate (ISD∧4/(hE)) is 1×10⁴ μm³/GPa or less.

Preferably, the ratio of the inter-spacing distance of the spacers to the average width of the spacer is 2 or larger.

Preferably, the ratio of the inter-spacing distance of the spacers to the average width of the spacer is 2 or larger, and the Young's modulus of the spacers multiplied by the filling factor of the spacers is at least 2 MPa.

Preferably, the inter-spacer distance that is at least about 2 times larger than the size of the analyte, up to 200 μm.

Preferably, there is provided a ratio of the inter-spacer-distance to the spacer width is 1.5 or larger.

Preferably, there is provided a ratio of the width to the height of the spacer is 1 or larger. More preferably, there is provided a ratio of the width to the height of the spacer is 1.5 or larger.

Preferably, the ratio of the width to the height of the spacer is 2 or larger. More preferably, the ratio of the width to the height of the spacer is larger than 2, 3, 5, 10, 20, 30, 50, or in a range of any two the value.

Preferably, the force that presses the two plates into the closed configuration is an imprecise pressing force. Preferably, the force that presses the two plates into the closed configuration is an imprecise pressing force provided by human hand. Preferably, the forcing of the two plates to compress at least part of the sample into a layer of substantially uniform thickness comprises a use of a conformable pressing, either in parallel or sequentially, an area of at least one of the plates to press the plates together to a closed configuration, wherein the conformable pressing generates a substantially uniform pressure on the plates over the at least part of the sample, and the pressing spreads the at least part of the sample laterally between the sample contact surfaces of the plates, and wherein the closed configuration is a configuration in which the spacing between the plates in the layer of uniform thickness region is regulated by the spacers; and wherein the reduced thickness of the sample reduces the time for mixing the reagents on the storage site with the sample.

Preferably, the pressing force is an imprecise force that has a magnitude which is, at the time that the force is applied, either (a) unknown and unpredictable, or (b) cannot be known and cannot be predicted within an accuracy equal or better than 20% of the average pressing force applied. More preferably, the pressing force is an imprecise force that has a magnitude which is, at the time that the force is applied, either (a) unknown and unpredictable, or (b) cannot be known and cannot be predicted within an accuracy equal or better than 30% of the average pressing force applied.

Preferably, the pressing force is an imprecise force that has a magnitude which is, at the time that the force is applied, either (a) unknown and unpredictable, or (b) cannot be known and cannot be predicted within an accuracy equal or better than 30% of the average pressing force applied; and wherein the layer of highly uniform thickness has a variation in thickness uniform of 20% or less.

Preferably, the pressing force is an imprecise force that has a magnitude which cannot, at the time that the force is applied, be determined within an accuracy equal or better than 30%, 40%, 50%, 70%, 100%, 200%, 300%, 500%, 1,000%, 2,000%, or in a range between any of the two values.

Preferably, the flexible plate has a thickness of in the range of 10 μm to 200 μm.

Preferably, the flexible plate has a thickness of in the range of 20 μm to 100 μm. More preferably, the flexible plate has a thickness of in the range of 25 μm to 180 μm. More preferably, the flexible plate has a thickness of in the range of 200 μm to 260 μm. More preferably, the flexible plate has a thickness of equal to or less than 250 μm, 225 μm, 200 μm, 175 μm, 150 μm, 125 μm, 100 μm, 75 μm, 50 μm, 25 μm, 10 μm, 5 μm, 1 μm, or in a range between the two of the values.

Preferably, the sample has a viscosity in the range of 0.1 to 4 (mPa s).

Preferably, the flexible plate has a thickness of in the range of 200 μm to 260 μm. More preferably, the flexible plate has a thickness in the range of 20 μm to 200 μm and Young's modulus in the range 0.1 to 5 GPa.

In one embodiment, the sample deposition of step (b) is a deposition directly from a subject to the plate without using any transferring devices. In one embodiment, during the deposition of step (b), the amount of the sample deposited on the plate is unknown.

In one embodiment, the method further comprises a analyzing step (e) that analyze the sample.

In one embodiment, the analyzing step (e) comprises calculating the volume of a relevant sample volume by measuring the lateral area of the relevant sample volume and calculating the volume from the lateral area and the predetermined spacer height.

In one embodiment, the analyzing step (e) comprises measuring: (i) imaging, (ii) illuminescence selected from photoluminescence, electroluminescence, and electrochemiluminescence, (iii) surface Raman scattering, (iv) electrical impedance selected from resistance, capacitance, and inductance, or (v) any combination of i-iv.

Preferably, the analyzing step (e) comprises reading, image analysis, or counting of the analyte, or a combination of thereof.

In one embodiment, the sample contains one or plurality of analytes, and one or both plate sample contact surfaces comprise one or a plurality of binding sites that each bind and immobilize a respective analyte.

In one embodiment, one or both plate sample contact surfaces comprise one or a plurality of storage sites that each stores a reagent or reagents, wherein the reagent(s) dissolve and diffuse in the sample during or after step (c).

In one embodiment, one or both plate sample contact surfaces comprises one or a plurality of amplification sites that are each capable of amplifying a signal from the analyte or a label of the analyte when the analyte or label is within 500 nm from an amplification site.

In one embodiment, there is provided: (i) one or both plate sample contact surfaces comprise one or a plurality of binding sites that each binds and immobilize a respective analyte; or (ii) one or both plate sample contact surfaces comprise, one or a plurality of storage sites that each stores a reagent or reagents; wherein the reagent(s) dissolve and diffuse in the sample during or after step (c), and wherein the sample contains one or plurality of analytes; or (iii) one or a plurality of amplification sites that are each capable of amplifying a signal from the analyte or a label of the analyte when the analyte or label is 500 nm from the amplification site; or (iv) any combination of (i) to (iii).

In one embodiment, the liquid sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

In one embodiment, the layer of uniform thickness in the closed configuration is less than 150 μm.

In one embodiment, the pressing is provided by a pressured liquid, a pressed gas, or a conformal material.

In one embodiment, the analyzing comprises counting cells in the layer of uniform thickness.

In one embodiment, the analyzing comprises performing an assay in the layer of uniform thickness.

In one embodiment, the assay is a binding assay or biochemical assay.

In one embodiment, the sample deposited has a total volume less 0.5 μL In one embodiment, multiple drops of sample are deposited onto one or both of the plates.

In one embodiment, the inter-spacer distance is in the range of 1 □m to 120 □m. In one embodiment, the inter-spacer distance is in the range of 120 □m to 50 □m.

In one embodiment, the inter-spacer distance is in the range of 120 □m to 200 □m.

In one embodiment, the flexible plates have a thickness in the range of 20 μm to 250 μm and Young's modulus in the range 0.1 to 5 GPa.

In one embodiment, for a flexible plate, the thickness of the flexible plate times the Young's modulus of the flexible plate is in the range 60 to 750 GPa-μm.

In one embodiment, the layer of uniform thickness sample is uniform over a lateral area that is at least 1 mm².

In one embodiment, the layer of uniform thickness sample is uniform over a lateral area that is at least 3 mm². Preferably, the layer of uniform thickness sample is uniform over a lateral area that is at least 5 mm². Preferably, the layer of uniform thickness sample is uniform over a lateral area that is at least 10 mm². More preferably, the layer of uniform thickness sample is uniform over a lateral area that is at least 20 mm².

In one embodiment, the layer of uniform thickness sample is uniform over a lateral area that is in a range of 20 mm² to 100 mm².

In one embodiment, the layer of uniform thickness sample has a thickness uniformity of up to +/−5% or better. Preferably, the layer of uniform thickness sample has a thickness uniformity of up to +1-10% or better. Preferably, the layer of uniform thickness sample has a thickness uniformity of up to +/−20% or better. Preferably, the layer of uniform thickness sample has a thickness uniformity of up to +/−30% or better. Preferably, the layer of uniform thickness sample has a thickness uniformity of up to +/−40% or better. Preferably, the layer of uniform thickness sample has a thickness uniformity of up to +/−50% or better.

In one embodiment, the spacers are pillars with a cross-sectional shape selected from round, polygonal, circular, square, rectangular, oval, elliptical, or any combination of the same.

In one embodiment, the spacers have pillar shape, have a substantially flat top surface, and have substantially uniform cross-section, wherein, for each spacer, the ratio of the lateral dimension of the spacer to its height is at least 1.

In one embodiment, the inter spacer distance is periodic.

In one embodiment, the spacers have a filling factor of 1% or higher, wherein the filling factor is the ratio of the spacer contact area to the total plate area.

In one embodiment, the Young's modulus of the spacers times the filling factor of the spacers is equal or larger than 20 MPa, wherein the filling factor is the ratio of the spacer contact area to the total plate area.

In one embodiment, the spacing between the two plates at the closed configuration is in less 200 μm.

In one embodiment, the spacing between the two plates at the closed configuration is a value selected from between 1.8 μm and 3.5 μm.

In one embodiment, the spacing are fixed on a plate by directly embossing the plate or injection molding of the plate.

In one embodiment, the materials of the plate and the spacers are selected from polystyrene, PMMA, PC, COC, COP, or another plastic.

In one embodiment, the spacers have a pillar shape, and the sidewall corners of the spacers have a round shape with a radius of curvature at least 1 □m.

In one embodiment, the spacers have a density of at least 1,000/mm².

In one embodiment, at least one of the plates is transparent.

In one embodiment, the mold used to make the spacers is fabricated by a mold containing features that are fabricated by either (a) directly reactive ion etching or ion beam etched or (b) by a duplication or multiple duplication of the features that are reactive ion etched or ion beam etched.

In one embodiment, the spacers are configured, such that the filling factor is in the range of 1% to 5%.

In one embodiment, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

In one embodiment, the spacers are configured, such that the filling factor is in the range of 1% to 5%. Preferably, the spacers are configured, such that the filling factor is in the range of 5% to 10%. Preferably, the spacers are configured, such that the filling factor is in the range of 10% to 20%. Preferably, the spacers are configured, such that the filling factor is in the range of 20% to 30%. Preferably, the spacers are configured, such that the filling factor is 5%, 10%, 20%, 30%, 40%, 50%, or in a range of any two of the values. Preferably, the spacers are configured, such that the filling factor is 50%, 60%, 70%, 80%, or in a range of any two of the values.

In one embodiment, the spacers are configured, such that the filling factor multiplies the Young's modulus of the spacer is in the range of 2 MPa and 10 MPa. Preferably, the spacers are configured, such that the filling factor multiplies the Young's modulus of the spacer is in the range of 10 MPa and 20 MPa. Preferably, the spacers are configured, such that the filling factor multiplies the Young's modulus of the spacer is in the range of 20 MPa and 40 MPa. Preferably, the spacers are configured, such that the filling factor multiplies the Young's modulus of the spacer is in the range of 40 MPa and 80 MPa. Preferably, the spacers are configured, such that the filling factor multiplies the Young's modulus of the spacer is in the range of 80 MPa and 120 MPa. Preferably, the spacers are configured, such that the filling factor multiplies the Young's modulus of the spacer is in the range of 120 MPa to 150 MPa.

In one embodiment, the device further comprises a dry reagent coated on one or both plates.

In one embodiment, the device further comprises, on one or both plates, a dry binding site that has a predetermined area, wherein the dry binding site binds to and immobilizes an analyte in the sample.

In one embodiment, the device further comprises, on one or both plates, a releasable dry reagent and a release time control material that delays the time that the releasable dry regent is released into the sample.

In one embodiment, the release time control material delays the time that the dry regent starts is released into the sample by at least 3 seconds.

In one embodiment, the regent comprises anticoagulant and/or staining reagent(s)

In one embodiment, the reagent comprises cell lysing reagent(s)

In one embodiment, the device further comprises, on one or both plates, one or a plurality of dry binding sites and/or one or a plurality of reagent sites.

In one embodiment, the analyte comprises a molecule (e.g., a protein, peptides, DNA, RNA, nucleic acid, or other molecule), cells, tissues, viruses, and nanoparticles with different shapes.

In one embodiment, the analyte comprises white blood cells, red blood cells and platelets. Preferably, the analyte is stained.

In one embodiment, the spacers regulating the layer of uniform thickness have a filling factor of at least 1%, wherein the filling factor is the ratio of the spacer area in contact with the layer of uniform thickness to the total plate area in contact with the layer of uniform thickness.

In one embodiment, for spacers regulating the layer of uniform thickness, the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness.

In one embodiment, for a flexible plate, the thickness of the flexible plate times the Young's modulus of the flexible plate is in the range 60 to 750 GPa-um.=

In one embodiment, for a flexible plate, the fourth power of the inter-spacer-distance (ISD) divided by the thickness of the flexible plate (h) and the Young's modulus (E) of the flexible plate, ISD⁴/(hE), is equal to or less than 10⁶ μm³/GPa,

In one embodiment, one or both plates comprise a location marker, either on a surface of or inside the plate, that provide information of a location of the plate.

In one embodiment, one or both plates comprise a scale marker, either on a surface of or inside the plate, that provide information of a lateral dimension of a structure of the sample and/or the plate.

In one embodiment, one or both plates comprise an imaging marker, either on surface of or inside the plate, that assists an imaging of the sample.

In one embodiment, the spacers functions as a location marker, a scale marker, an imaging marker, or any combination of thereof.

In one embodiment, the average thickness of the layer of uniform thickness is about equal to a minimum dimension of an analyte in the sample.

In one embodiment, the inter-spacer distance is in the range of 7 μm to 50 μm. Preferably, the inter-spacer distance is in the range of 50 μm to 120 μm. Preferably, the inter-spacer distance is in the range of 120 μm to 200 μm (micron).

In one embodiment, the inter-spacer distance is substantially periodic.

In one embodiment, the spacers are pillars with a cross-sectional shape selected from round, polygonal, circular, square, rectangular, oval, elliptical, or any combination of the same.

In one embodiment, the spacers have are pillar shape and have a substantially flat top surface. In one embodiment, for each spacer, the ratio of the lateral dimension of the spacer to its height is at least 1.

In one embodiment, the minimum lateral dimension of spacer is less than or substantially equal to the minimum dimension of an analyte in the sample. Preferably the minimum lateral dimension of spacer is in the range of 0.5 μm to 100 μm. Preferably, the minimum lateral dimension of spacer is in the range of 0.5 μm to 10 μm.

In one embodiment, the sample is blood. In one embodiment, the sample is whole blood without dilution by liquid. In another embodiment, the sample is a diluted blood.

In one embodiment, the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

In one embodiment, the sample is a biological sample, an environmental sample, a chemical sample, or clinical sample.

In one embodiment, the spacers have a pillar shape, and the sidewall corners of the spacers have a round shape with a radius of curverture at least 1 μm.

In one embodiment, the spacers have a density of at least 100/mm². Preferably, the spacers have a density of at least 1,000/mm².

In one embodiment, at least one of the plates is transparent.

In one embodiment, one of the plates is made from a flexible polymer.

In one embodiment, for a pressure that compresses the plates, the spacers are not compressible and/or, independently, only one of the plates is flexible.

In one embodiment, the flexible plate has a thickness in the range of 10 μm to 200 μm.

In one embodiment, the variation is less than 30%. In one embodiment, the variation is less than 10%. In one embodiment, the variation is less than 5%.

In one embodiment, the first and second plates are connected and are configured to be changed from the open configuration to the closed configuration by folding the plates.

In one embodiment, the first and second plates are connected by a hinge and are configured to be changed from the open configuration to the closed configuration by folding the plates along the hinge.

In one embodiment, the first and second plates are connected by a hinge that is a separate material to the plates, and are configured to be changed from the open configuration to the closed configuration by folding the plates along the hinge

In one embodiment, the first and second plates are made in a single piece of material and are configured to be changed from the open configuration to the closed configuration by folding the plates.

In one embodiment, the layer of uniform thickness sample is uniform over a lateral area that is at least 1 mm².

In one embodiment, the device is configured to analyze the sample in 60 seconds or less.

In one embodiment, at the closed configuration, the final sample thickness device is configured to analyze the sample in 60 seconds or less.

In one embodiment, at the closed configuration, the final sample thickness device is configured to analyze the sample in 10 seconds or less.

In one embodiment, the dry binding site comprises a capture agent. In one embodiment, the dry binding site comprises an antibody or nucleic acid.

In one embodiment, the releasable dry reagent is a labeled reagent. In one embodiment, the releasable dry reagent is a fluorescently-labeled reagent. In one embodiment, the releasable dry reagent is a fluorescently-labeled antibody. In one embodiment, the releasable dry reagent is a cell stain. In one embodiment, the releasable dry reagent is a cell lysing.

In one embodiment, the detector is an optical detector that detects an optical signal. In one embodiment, the detector is an electric detector that detect electrical signal.

In one embodiment, the spacing are fixed on a plate by directly embossing the plate or injection molding of the plate.

In one embodiment, the materials of the plate and the spacers are selected from polystyrene, PMMA, PC, COC, COP, or another plastic.

In one aspect, there is provided a system for rapidly analyzing a sample using a mobile phone comprising:
(a) a device of any prior embodiment;
(b) a mobile communication device comprising:
   i. one or a plurality of cameras for the detecting and/or imaging the sample;
   ii. electronics, signal processors, hardware and software for receiving and/or processing the detected signal and/or the image of the sample and for remote communication; and
(c) a light source from either the mobile communication device or an external source;
wherein the detector in the devices or methods of any prior embodiment is provided by the mobile communication device, and detects an analyte in the sample at the closed configuration.

In one embodiment, one of the plates has a binding site that binds an analyte, wherein at least part of the uniform sample thickness layer is over the binding site, and is substantially less than the average lateral linear dimension of the binding site.

In one embodiment, the present system further comprising: (d) a housing configured to hold the sample and to be mounted to the mobile communication device.

In one embodiment, the housing comprises optics for facilitating the imaging and/or signal processing of the sample by the mobile communication device, and a mount configured to hold the optics on the mobile communication device.

In one embodiment, an element of the optics in the housing is movable relative to the housing.

In one embodiment, the mobile communication device is configured to communicate test results to a medical professional, a medical facility or an insurance company. In one embodiment, the mobile communication device is further configured to communicate information on the test and the subject with the medical professional, medical facility or insurance company.

In one embodiment, the mobile communication device is further configured to communicate information of the test to a cloud network, and the cloud network process the information to refine the test results. In one embodiment, the mobile communication device is further configured to communicate information of the test and the subject to a cloud network, the cloud network processes the information to refine the test results, and the refined test results will send back the subject.

In one embodiment, the mobile communication device is configured to receive a prescription, diagnosis or a recommendation from a medical professional.

In one embodiment, the mobile communication device is configured with hardware and software to:
(a) capture an image of the sample;
(b) analyze a test location and a control location in in image; and
(c) compare a value obtained from analysis of the test location to a threshold value that characterizes the rapid diagnostic test.

In one embodiment, at least one of the plates comprises a storage site in which assay reagents are stored.

In one embodiment, at least one of the cameras reads a signal from the device.

In one embodiment, the mobile communication device communicates with the remote location via a WIFI or cellular network.

In one embodiment, the mobile communication device is a mobile phone.

In one aspect, there is provided a method for rapidly analyzing an analyte in a sample using a mobile phone, comprising:

(a) depositing a sample on the device of any prior system embodiment;

(b) assaying an analyte in the sample deposited on the device to generate a result; and

(c) communicating the result from the mobile communication device to a location remote from the mobile communication device.

In one embodiment, the analyte comprises a molecule (e.g., a protein, peptides, DNA, RNA, nucleic acid, or other molecule), cells, tissues, viruses, and nanoparticles with different shapes. In one embodiment, the analyte comprises white blood cell, red blood cell and platelets.

In one embodiment, the assaying comprises performing a white blood cells differential assay.

In one embodiment, the present method comprises:
(a) analyzing the results at the remote location to provide an analyzed result; and
(b) communicating the analyzed result from the remote location to the mobile communication device.

In one embodiment, the analysis is done by a medical professional at a remote location.

In one embodiment, the mobile communication device receives a prescription, diagnosis or a recommendation from a medical professional at a remote location.

In one embodiment, the sample is a bodily fluid. In one embodiment, the bodily fluid is blood, saliva or urine. In one embodiment, the sample is whole blood without dilution by a liquid.

In one embodiment, the assaying step comprises detecting an analyte in the sample.

In one embodiment, the analyte is a biomarker. In one embodiment, the analyte is a protein, nucleic acid, cell, or metabolite.

In one embodiment, the present method comprises counting the number of red blood cells. In one embodiment, the present method comprises counting the number of white blood cells. In one embodiment, the present method comprises staining the cells in the sample and counting the number of neutrophils, lymphocytes, monocytes, eosinophils and basophils.

In one embodiment, the present assay performed in step (b) is a binding assay or a biochemical assay.

In one aspect, there is provided a method for analyzing a sample comprising:
(a) obtaining a device of any prior device embodiment;
(b) depositing the sample onto one or both pates of the device;
(c) placing the plates in a closed configuration and applying an external force over at least part of the plates; and
(d) analyzing the in the layer of uniform thickness while the plates are the closed configuration.

In one embodiment, the first plate further comprises, on its surface, a first predetermined assay site and a second predetermined assay site, wherein the distance between the edges of the assay site is substantially larger than the thickness of the uniform thickness layer when the plates are in the closed position, wherein at least a part of the uniform thickness layer is over the predetermined assay sites, and wherein the sample has one or a plurality of analytes that are capable of diffusing in the sample.

In one embodiment, the first plate has, on its surface, at least three analyte assay sites, and the distance between the edges of any two neighboring assay sites is substantially larger than the thickness of the uniform thickness layer when the plates are in the closed position, wherein at least a part of the uniform thickness layer is over the assay sites, and wherein the sample has one or a plurality of analytes that are capable of diffusing in the sample.

In one embodiment, the first plate has, on its surface, at least two neighboring analyte assay sites that are not separated by a distance that is substantially larger than the thickness of the uniform thickness layer when the plates are in the closed position, wherein at least a part of the uniform thickness layer is over the assay sites, and wherein the sample has one or a plurality of analytes that are capable of diffusing in the sample.

In one embodiment, the analyte assay area is between a pair of electrodes.

In one embodiment, the assay area is defined by a patch of dried reagent.

In one embodiment, the assay area binds to and immobilizes the analyte

In one embodiment, the assay area is defined by a patch of binding reagent that, upon contacting the sample, dissolves into the sample, diffuses in the sample, and binds to the analyte.

In one embodiment, the inter-spacer distance is in the range of 14 □m to 200 □m. In one embodiment, the inter-spacer distance is in the range of 7 □m to 20 □m.

In one embodiment, the spacers are pillars with a cross-sectional shape selected from round, polygonal, circular, square, rectangular, oval, elliptical, or any combination of the same.

In one embodiment, the spacers have are pillar shape and have a substantially flat top surface, wherein, for each spacer, the ratio of the lateral dimension of the spacer to its height is at least 1.

In one embodiment, the spacers have a pillar shape, and the sidewall corners of the spacers have a round shape with a radius of curverture at least 1 □m.

In one embodiment, the spacers have a density of at least 1,000/mm².

In one embodiment, at least one of the plates is transparent.

In one embodiment, at least one of the plates is made from a flexible polymer.

In one embodiment, only one of the plates is flexible.

In one embodiment, the area-determination device is a camera.

In one embodiment,]the area-determination device comprises an area in the sample contact area of a plate, wherein the area is less than 1/100, 1/20, 1/10, ⅙, ⅕, ¼, ⅓, ½, ⅔ of the sample contact area, or in a range between any of the two values.

In one embodiment, he area-determination device comprises a camera and an area in the sample contact area of a plate, wherein the area is in contact with the sample.

In one embodiment, the deformable sample comprises a liquid sample.

In one embodiment, the imprecision force has a variation at least 30% of the total force that actually is applied.

In one embodiment, the imprecision force has a variation at least 20%, 30%, 40%, 50%, 60, 70%, 80%, 90% 100%, 150%, 200%, 300%, 500%, or in a range of any two values, of the total force that actually is applied.

In one embodiment, the spacers have a flat top.

In one embodiment, the device is further configured to have, after the pressing force is removed, a sample thickness that is substantially the same in thickness and uniformity as that when the force is applied.

In one embodiment, the imprecise force is provided by human hand.

In one embodiment, the inter spacer distance is substantially constant. In one embodiment, the inter spacer distance is substantially periodic in the area of the uniform sample thickness area.

In one embodiment, the multiplication product of the filling factor and the Young's modulus of the spacer is 2 MPa or larger.

In one embodiment, the force is applied by hand directly or indirectly.

In one embodiment, the force applied is in the range of 1 N to 20 N. In one embodiment, the force applied is in the range of 20 N to 200 N.

In one embodiment, the highly uniform layer has a thickness that varies by less than 15%, 10%, or 5% of an average thickness.

In one embodiment, the imprecise force is applied by pinching the device between a thumb and forefinger.

In one embodiment, the predetermined sample thickness is larger than the spacer height.

In one embodiment, the device holds itself in the closed configuration after the pressing force has been removed.

In one embodiment, the uniform thickness sample layer area is larger than that area upon which the pressing force is applied.

In one embodiment, the spacers do not significantly deform during application of the pressing force.

In one embodiment, the pressing force is not predetermined beforehand and is not measured.

In some embodiments, the fluidic sample is replaced by a deformable sample and the embodiments for making at least a part of the fluidic sample into a uniform thickness layer can make at least a part of the deformable sample into a uniform thickness layer.

In one embodiment, the inter spacer distance is periodic.

In one embodiment, the spacers have a flat top.

In one embodiment, the inter spacer distance is at least two times large than the size of the targeted analyte in the sample.

In one aspect, there is provided a method of manufacturing a Q-Card.

In one embodiment, the Q-Card comprising: a first plate, a second plate, and a hinge, wherein
i. the first plate, that is about 200 nm to 1500 nm thick, comprises, on its inner surface, (a) a sample contact area for contacting a sample, and (b) a sample overflow dam that surrounds the sample contact area is configured to present a sample flow outside of the dam;
ii. the second plate is 10 um to 250 um thick and comprises, on its inner surface, (a) a sample contact area for contacting a sample, and (b) spacers on the sample contact area;
iii. the hinge that connect the first and the second plates; and
   wherein the first and second plate are movable relative to each other around the axis of the hinge.

In one embodiment, the Q-Card comprising: a first plate, a second plate, and a hinge, wherein
i. the first plate, that is about 200 nm to 1500 nm thick, comprises, on its inner surface, (a) a sample contact area for contacting a sample, (b) a sample overflow dam that surrounds the sample contact area is configured to present a sample flow outside of the dam, and (c) spacers on the sample contact area;
ii. the second plate, that is 10 um to 250 um thick, comprises, on its inner surface, a sample contact area for contacting a sample;
iii. the hinge that connect the first and the second plates; and
   wherein the first and second plate are movable relative to each other around the axis of the hinge.

In one embodiment, the Q-Card comprising: a first plate, a second plate, and a hinge, wherein
i. the first plate, that is about 200 nm to 1500 nm thick, comprises, on its inner surface, (a) a sample contact area for contacting a sample, and (b) spacers on the sample contact area;
ii. the second plate, that is 10 um to 250 um thick, comprises, on its inner surface, (a) a sample contact area for contacting a sample, and (b) a sample overflow dam that surrounds the sample contact area is configured to present a sample flow outside of the dam, and;
iii. the hinge that connect the first and the second plates; and
   wherein the first and second plate are movable relative to each other around the axis of the hinge.

In one embodiment, the Q-Card comprising: a first plate, a second plate, and a hinge, wherein
i. the first plate, that is about 200 nm to 1500 nm thick, comprises, on its inner surface, a sample contact area for contacting a sample;
ii. the second plate, that is 10 um to 250 um thick, comprises, on its inner surface, (a) a sample contact area for contacting a sample, (b) a sample overflow dam that surrounds the sample contact area is configured to present a sample flow outside of the dam, and (c) spacers on the sample contact area; and
iii. the hinge that connect the first and the second plates; and
   wherein the first and second plate are movable relative to each other around the axis of the hinge.

In one embodiment, there is provided a method for fabricating a Q-Card, comprising:
(a) injection molding of the first plate; and
(b) nanoimprinting or extrusion printing of the second plate.

In one embodiment, there is provide a method for fabricating a Q-Card, comprising:
(a) Laser cutting the first plate; and
(b) nanoimprinting or extrusion printing of the second plate.

In one embodiment, there is provided a method for fabricating a Q-Card, comprising:
(a) injection molding and laser cutting the first plate; and
(b) nanoimprinting or extrusion printing of the second plate.

In one embodiment, there is provided a method for fabricating a Q-Card, comprising: nanoimprinting or extrusion printing to fabricated both the first and the second plate.

In one embodiment, there is provided a method for fabricating a Q-Card, comprising: fabricating the first plate or the second plate, using injection molding, laser cutting the first plate, nanoimprinting, extrusion printing, or a combination of thereof.

In one embodiment, the method further comprises a step of attach the hinge on the first and the second plates after the fabrication of the first and second plates.

Compressed Regulated Open Flow” (CROF)

In assaying, a manipulation of a sample or a reagent can lead to improvements in the assaying. The manipulation includes, but not limited to, manipulating the geometric shape and location of a sample and/or a reagent, a mixing or a binding of a sample and a reagent, and a contact area of a sample of reagent to a plate.

Many embodiments of the present invention manipulate the geometric size, location, contact areas, and mixing of a sample and/or a reagent using a method, termed “compressed regulated open flow (CROF)”, and a device that performs CROF.

The term “compressed open flow (COF)” refers to a method that changes the shape of a flowable sample deposited on a plate by (i) placing other plate on top of at least a part of the sample and (ii) then compressing the sample between two plates by pushing the two plates towards each other; wherein the compression reduces a thickness of at least a part of the sample and makes the sample flow into open spaces between the plates.

The term “compressed regulated open flow” or “CROF” (or “self-calibrated compressed open flow” or “SCOF” or “SCCOF”) refers to a particular type of COF, wherein the final thickness of a part or entire sample after the compression is “regulated” by spacers, wherein the spacers, that are placed between the two plates.

The term “the final thickness of a part or entire sample is regulated by spacers” in a CROF means that during a CROF, once a specific sample thickness is reached, the relative movement of the two plates and hence the change of sample thickness stop, wherein the specific thickness is determined by the spacer.

In one embodiment, the method of CROF comprises:

(a) obtaining a sample, that is flowable;

(b) obtaining a first plate and a second plate that are movable relative to each other into different configurations, wherein each plate has a sample contact surface that is substantially planar, wherein one or both of the plates comprise spacers and the spacers have a predetermined height, and the spacers are on a respective sample contacting surface;

(c) depositing, when the plates are configured in an open configuration, the sample on one or both of the plates; wherein the open configuration is a configuration in which the two plates are either partially or completely separated apart and the spacing between the plates is not regulated by the spacers; and

(d) after (c), spreading the sample by bringing the plates into a closed configuration, wherein, in the closed configuration: the plates are facing each other, the spacers and a relevant volume of the sample are between the plates, the thickness of the relevant volume of the sample is regulated by the plates and the spacers, wherein the relevant volume is at least a portion of an entire volume of the sample, and wherein during the sample spreading, the sample flows laterally between the two plates.

(1) Hinges, Opening Notches, Recessed Edge and Sliders

The devices/apparatus, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/431,639, which was filed on Dec. 9, 2016, U.S. Provisional Application No. 62/456,065, which was filed on Feb. 7, 2017, U.S. Provisional Application Nos. 62/456,287 and 62/456,504, which was filed on Feb. 8, 2017, and U.S. Provisional Application No. 62/539,660, which was filed on Aug. 1, 2017, all of which applications are incorporated herein in their entireties for all purposes.

In some embodiments, the QMAX device comprises opening mechanisms such as but not limited to notches on plate edges or strips attached to the plates, making is easier for a user to manipulate the positioning of the plates, such as but not limited to separating the plates of by hand.

In some embodiments, the QMAX device comprises trenches on one or both of the plates. In certain embodiments, the trenches limit the flow of the sample on the plate.

(2) Q-Card and Adaptor

The devices/apparatus, systems, and methods herein disclosed can include or use Q-cards for sample detection, analysis, and quantification. In some embodiments, the Q-card is used together with an adaptor that is configured to accommodate the Q-card and connect to a mobile device so that the sample in the Q-card can be imaged, analyzed, and/or measured by the mobile device. The structure, material, function, variation, dimension and connection of the Q-card, the adaptor, and the mobile are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the adaptor comprises a receptacle slot, which is configured to accommodate the QMAX device when the device is in a closed configuration. In certain embodiments, the QMAX device has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the QMAX device and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample.

(3) Smartphone Detection System

The devices/apparatus, systems, and methods herein disclosed can include or use Q-cards for sample detection, analysis, and quantification. In some embodiments, the Q-card is used together with an adaptor that can connect the Q-card with a smartphone detection system. In some embodiments, the smartphone comprises a camera and/or an illumination source The smartphone detection system, as well the associated hardware and software are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the smartphone comprises a camera, which can be used to capture images or the sample when the sample is positioned in the field of view of the camera (e.g. by an adaptor). In certain embodiments, the camera includes one set of lenses (e.g. as in iPhone™ 6). In certain embodiments, the camera includes at least two sets of lenses (e.g. as in iPhone™ 7). In some embodiments, the smartphone comprises a camera, but the camera is not used for image capturing.

In some embodiments, the smartphone comprises a light source such as but not limited to LED (light emitting diode). In certain embodiments, the light source is used to provide illumination to the sample when the sample is positioned in the field of view of the camera (e.g. by an adaptor). In some embodiments, the light from the light source is enhanced, magnified, altered, and/or optimized by optical components of the adaptor.

In some embodiments, the smartphone comprises a processor that is configured to process the information from the sample. The smartphone includes software instructions that, when executed by the processor, can enhance, magnify, and/or optimize the signals (e.g. images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

In some embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

In some embodiments, the smartphone is an iPhone™, an Android™ phone, or a Wndows™ phone.

(4) Detection Methods

The devices/apparatus, systems, and methods herein disclosed can include or be used in various types of detection methods. The detection methods are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287, 62/456,528, 62/456,631, 62/456,522, 62/456,598, 62/456,603, and 62/456,628, which were filed on Feb. 8, 2017, U.S. Provisional Application Nos. 62/459,276, 62/456,904, 62/457,075, and 62/457,009, which were filed on Feb. 9, 2017, and U.S. Provisional Application Nos. 62/459,303, 62/459,337, and 62/459,598, which were filed on Feb. 15, 2017, and U.S. Provisional Application Nos. 62/460,083, 62/460,076, which were filed on Feb. 16, 2017, all of which applications are incorporated herein in their entireties for all purposes.

(5) Labels, Capture Agent and Detection Agent

The devices/apparatus, systems, and methods herein disclosed can employ various types of labels, capture agents, and detection agents that are used for analytes detection. The labels are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the label is optically detectable, such as but not limited to a fluorescence label. In some embodiments, the labels include, but are not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; 1R144; 1R1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

In any embodiment, the QMAX device can contain a plurality of capture agents and/or detection agents that each bind to a biomarker selected from Tables B1, B2, B3 and/or B7 in U.S. Provisional Application No. 62/234,538 and/or PCT Application No. PCT/US2016/054025, wherein the reading step d) includes obtaining a measure of the amount of the plurality of biomarkers in the sample, and wherein the amount of the plurality of biomarkers in the sample is diagnostic of a disease or condition.

In any embodiment, the capture agent and/or detection agents can be an antibody epitope and the biomarker can be an antibody that binds to the antibody epitope. In some embodiments, the antibody epitope includes a biomolecule, or a fragment thereof, selected from Tables B4, B5 or B6 in U.S. Provisional Application No. 62/234,538 and/or PCT Application No. PCT/US2016/054025. In some embodiments, the antibody epitope includes an allergen, or a fragment thereof, selected from Table B5. In some embodiments, the antibody epitope includes an infectious agent-derived biomolecule, or a fragment thereof, selected from Table B6 in U.S. Provisional Application No. 62/234,538 and/or PCT Application No. PCT/US2016/054025.

In any embodiment, the QMAX device can contain a plurality of antibody epitopes selected from Tables B4, B5 and/or B6 in U.S. Provisional Application No. 62/234,538 and/or PCT Application No. PCT/US2016/054025, wherein the reading step d) includes obtaining a measure of the amount of a plurality of epitope-binding antibodies in the sample, and wherein the amount of the plurality of epitope-binding antibodies in the sample is diagnostic of a disease or condition.

(6) Analytes

The devices/apparatus, systems, and methods herein disclosed can be applied to manipulation and detection of various types of analytes (including biomarkers). The analytes are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

Also provided herein are kits that find use in practicing the devices, systems and methods in the present invention.

The amount of sample can be about a drop of a sample. The amount of sample can be the amount collected from a pricked finger or fingerstick. The amount of sample can be the amount collected from a microneedle or a venous draw.

A sample can be used without further processing after obtaining it from the source, or can be processed, e.g., to enrich for an analyte of interest, remove large particulate matter, dissolve or resuspend a solid sample, etc.

Any suitable method of applying a sample to the QMAX device can be employed. Suitable methods can include using a pipet, dropper, syringe, etc. In certain embodiments, when the QMAX device is located on a support in a dipstick format, as described below, the sample can be applied to the QMAX device by dipping a sample-receiving area of the dipstick into the sample.

A sample can be collected at one time, or at a plurality of times. Samples collected over time can be aggregated and/or processed (by applying to a QMAX device and obtaining a measurement of the amount of analyte in the sample, as described herein) individually. In some instances, measurements obtained over time can be aggregated and can be useful for longitudinal analysis over time to facilitate screening, diagnosis, treatment, and/or disease prevention.

Washing the QMAX device to remove unbound sample components can be done in any convenient manner, as described above. In certain embodiments, the surface of the QMAX device is washed using binding buffer to remove unbound sample components.

Detectable labeling of the analyte can be done by any convenient method. The analyte can be labeled directly or indirectly. In direct labeling, the analyte in the sample is labeled before the sample is applied to the QMAX device. In indirect labeling, an unlabeled analyte in a sample is labeled after the sample is applied to the QMAX device to capture the unlabeled analyte, as described below.

(7) Applications

The devices/apparatus, systems, and methods herein disclosed can be used for various applications (fields and samples). The applications are herein disclosed, listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

What is claimed is:

1. A method, comprising the steps of:
(a) providing a device comprising a first plate and a second plate, said first plate and/or said second plate comprises, on its inner surface, a sample contact area that is configured to contact a sample, wherein the sample contains or suspected of containing bacteria that cause sexually transmitted diseases (STDs);
(b) depositing the sample onto the sample contact area;
(c) adding a staining medium to the deposited sample to form a mixture, wherein the staining medium comprises an antibody specific to the bacteria;
(d) compressing the first plate with the second plate so that at least part of the mixture forms a thin layer;
(e) incubating the mixture so that the antibody binds to the STD-causing bacteria and yields a signal; and
(f) detecting the signal, wherein the detected signal is indicative of the presence of bacteria in the sample.

2. The method of any prior claim, wherein the bacteria that cause STD is selected from the group consisting of Chlamydia trachomatis, Neisseria gonorrhoeae, and Treponema pallidium.

3. The method of any prior claim, wherein the incubating step performed for about 60 seconds or less.

4. The method of any prior claim, wherein the incubating step performed for about 30 seconds or less.

5. The method of any prior claim, wherein the incubating step is performed for about 15 seconds or less.

6. The method of any prior claim, wherein the antibody is fluorescently labeled.

7. The method of any prior claim, wherein the thin layer has a uniform thickness that is less than 100 μm.

8. The method of any prior claim, wherein the thin layer has a uniform thickness that is less than 50 μm.

9. The method of any prior claim, wherein the thin layer has a uniform thickness that is about 30 μm or less.

10. The method of any prior claim, wherein the signal is detected by imaging the incubated sample step (f).

11. A method, comprising the steps of:
(a) providing a device comprising a first plate and a second plate, one or both of the plates comprises, on its inner surface, a sample contact area that has a binding site,
wherein the sample contact area is configured to contact a sample,
wherein the sample contains or suspected of comprising bacteria that cause sexually transmitted diseases (STDs), and
wherein the binding site comprises an immobilized capture antibody that binds to the bacteria in the sample;
(b) providing one or both of the plates comprising, on its inner surface, a sample contact area that has a storage site,
wherein the storage site comprises a detection antibody that is capable of, upon contacting the sample, diffusing in the sample, and
wherein the capture antibody and detection antibody bind to different sites on the bacteria to form a capture antibody-bacteria-detection antibody sandwich;
(c) depositing the sample onto one or both of the sample contact areas of the plates;
(d) bringing the two plates to a closed configuration, wherein, in the closed configuration, at least part of the deposited sample in (c) is confined between the sample contact areas of the two plates, and the first plate and the second plate has an average thickness in the range of 0.01 μm to 200 μm; and
(e) detecting a signal, wherein the signal is generated after the capture antibody-bacteria-detection antibody is formed, and the detected signal is indicative of the presence of bacteria that cause sexually transmitted diseases (STDs) in the sample.

12. The method of any prior claim, wherein the sample is from a human.

13. The method of any prior claim, wherein the bacteria is selected from the group consisting of Chlamydia trachomatis, Neisseria gonorrhoeae, and Treponema pallidium.

14. The method of any prior claim, wherein the capture antibody has a capturing site that comprises a protein stabilizer.

15. The method of any prior claim, wherein the storage site further comprises a protein stabilizer.

16. The method of any prior claim, wherein the detection antibody comprises a fluorescent label.

17. The method of any prior claim, wherein the sample between the two plates has a uniform thickness in the range of 0.5 μm to 50 μm.

18. The method of any prior claim, wherein the sample between the two plates has a uniform thickness in the range of 1 μm to 35 μm.

19. The method of any prior claim, further comprising the step (g): determining the presence or absence of STD-causing bacteria.

20. The method of any prior claim, wherein the steps (a)-(e) are performed in less than 10 minutes.

21. The method of any prior claim, wherein the steps (e)-(e) are performed in less than 3 minutes.

22. The method of any prior claim, wherein the steps (a)-(e) are performed in less than 2 minutes.

23. The method of any prior claim, wherein one or both of the sample contact areas comprise a plurality of spacers, wherein the plurality of spacers regulate the spacing between the sample contact areas of the plates when the plates are in the closed configuration.

24. The method of any prior claim, wherein the first plate comprises a plurality of binding sites and the second plate comprises a plurality of corresponding storage sites, wherein each binding site faces a corresponding storage site when the plates are in the closed configuration.

25. The method and device of any prior embodiment, wherein the detection antibody is dried on the storage site.

26. The method of any prior claim, wherein the capture antibody at the binding site is on an amplification surface that amplifies an optical signal of the captured detection antibody.

27. The method of any prior claim, wherein the capture antibody at the binding site are on an amplification surface that amplifies an optical signal of the captured detection antibody, wherein the amplification is proximity-dependent in that the amplification significantly reduced as the distance between the capture antibody and the detection antibody increases.

28. The method of any prior claim, wherein the signal is detected by electrical means, optical means, or both.

29. The method of any prior claim, wherein the signal is detected by fluorescence or SPR.

30. A method, comprising the steps of:
(a) providing a device comprising a first plate and a second plate, one or both of the plates comprises, on its inner surface, a sample contact area that has a binding site,
wherein the sample contact area is configured to contact a liquid sample,
wherein the liquid sample contains or is suspected of containing cells that express a biomarker,
(b) providing one or both of the plates comprising, on its inner surface, a sample contact area that has a storage site,
wherein the storage site comprises a detection agent positioned therein,
wherein the detection agent is configured to bind to the biomarker,
(c) depositing the sample onto one or both of the sample contact areas of the plates;
wherein the deposited liquid sample is in contact with the detecting agent;
(d) bringing the two plates to a closed configuration, wherein, in the closed configuration, at least part of the deposited sample in (c) is confined between the sample contact areas of the two plates, and the first plate and the second plate has an average thickness in the range of 0.01 μm to 200 μm;
(e) incubating the deposited liquid sample for a period of time;
(f) quantifying the cells expressing the biomarker by imaging the deposited sample layer and counting the cells that expresses the biomarker.

31. A method, comprising:
(a) providing a first plate and a second plate, wherein each plate comprises, on its respective inner surface, a sample contact area that is configured to contact a liquid sample,
wherein a detecting agent is positioned on the sample contact area of one or both of the plates, and
wherein the detecting agent is configured to specifically bind to the biomarker;
(b) depositing the sample onto the sample contact area,
wherein the deposited sample comprises the cell that expresses the biomarker;
(c) pressing the first plate and the second plate to compress the deposited sample into a thin layer, which is at least partly confined by the two sample contact areas that face each other;
(d) incubating for a period of time that is about 60 seconds or less; and
(e) quantifying the cell expressing the biomarker by imaging the deposited sample layer and counting the cell expressing the biomarker.

32. A method, comprising:
(a) providing a first plate and a second plate, each plate comprises, on its respective inner surface, a sample contact area that is configured to contact a blood sample,
wherein a detecting agent is positioned on the sample contact area of one or both of the plates, and
wherein the detecting agent is configured to specifically bind to an antigen selected from the group consisting of CD3, CD4 and CD8,
(b) depositing the blood sample in the sample contact area, wherein the blood sample comprises cells that express CD3, CD4, or CD8;
(c) pressing the first plate and the second plate to compress the blood sample into a thin layer, which is at least partly confined by the two sample contact areas that face each other;
(d) incubating for a period of time that is about 60 seconds or less; and
(e) quantifying the cells expressing CD3, CD4 or CD8 by imaging the compressed blood sample and counting the cells expressing CD3, CD4 or CD8.

33. An apparatus, comprising:
(a) a first plate and a second plate, movable relative to each other into different configurations, including a closed configuration and an open configuration, wherein each plate comprises, on its respective inner surface, a sample contact area that is configured to contact a liquid sample that expresses or is expected to express a biomarker,
wherein a detecting agent is positioned on one or both of the plates and is configured to specifically bind to the biomarker; and
(b) an adaptor that is configured to accommodate the first plate and second plate when in a closed configuration and be attachable to a mobile device, wherein:
i. the mobile device comprises an imager,
ii. the adaptor is configured to position the liquid sample in a field of view (FOV) of the imager when the adaptor is attached to the mobile device, and
iii. the imager is configured to capture images of the liquid sample, thereby detecting/measuring a signal that is generated by the binding of the biomarker with the detecting agent after the sample is incubated with the detecting agent for a period of time that is about 60 seconds or less.

34. The method or apparatus of any prior claim, wherein the period of time that is about 30 seconds or less.

35. The method or apparatus of any prior claim, with the proviso that the sample contact areas are not washed after the incubating step (d).

36. The method or apparatus of any prior claim, wherein the detecting agent is an antibody.

37. The method or apparatus of any prior claim, wherein the antibody is labeled with a fluorophore.

38. The method or apparatus of any prior claim, wherein the detecting agent is labeled with a signaling molecule that emits a signal upon excitation.

39. The method or apparatus of any prior claim, wherein the thin layer has a uniform thickness that is about equal to or less than 10 μm.

40. The method or apparatus of any prior claim, wherein the thin layer has a uniform thickness that is about equal to or less than 2 μm.

41. The method or apparatus of any prior claim, wherein the sample is whole blood.

42. The method or apparatus of any prior claim, wherein the biomarker is CD3 (cluster of differentiation 3).

43. The method or apparatus of any prior claim, wherein the biomarker is CD4 (cluster of differentiation 4).

44. The method or apparatus of any prior claim, wherein the biomarker is CD8 (cluster of differentiation 8).

45. The method or apparatus of any prior claim, wherein the cells are T cells.

46. The method or apparatus of any prior claim, wherein the detecting agent is immobilized on the sample contact area.

47. The method or apparatus of any prior claim, wherein the stained cell or bacteria is imaged without washing away the staining solution.

48. The method or apparatus of any prior claim, wherein the stained cell or bacteria is imaged with one step washing of the staining solution.

49. The method or apparatus of any prior claim, wherein the one step washing is performed by washing the binding site for 1 minute.

50. The method or apparatus of any prior claim, wherein the one step washing is performed by washing the binding site for 1 minute and then watering.
